(19) United States
(12) Patent Application Publication
(10) Pub. No.: US 2025/0236596 A1
(43) Pub. Date: Jul. 24, 2025

(54) HYDROXYLAMINE-BASED EGFR INHIBITORS FOR TREATMENT OF CANCER WITH BRAIN METASTASES

(71) Applicant: University of Georgia Research Foundation, Inc., Athens, GA (US)

(72) Inventors: David Crich, Athens, GA (US); Jarvis Dawson Hill, New Haven, CT (US)

(21) Appl. No.: 19/110,836

(22) PCT Filed: Sep. 13, 2023
(86) PCT No.: PCT/US2023/074118

§ 371 Date: Mar. 12, 2025

Related U.S. Application Data
(60) Provisional application No. 63/507,876, filed on Jun. 13, 2023.
(60) Provisional application No. 63/375,622, filed on Sep. 14, 2022.

(51) Int. Cl. C07D 239/94; A61K 31/517; A61K 31/5377; A61P 35/04; C07D 401/12

(52) U.S. Cl. CPC C07D 239/94; A61K 31/517; A61K 31/5377; A61P 35/04; C07D 401/12

(57) ABSTRACT

In accordance with the purpose(s) of the present disclosure, as embodied and broadly described herein, the disclosure, in one aspect, relates to scaffold molecules that inhibit epidermal growth factor receptor (EGFR), the methods of making same, the pharmaceutical compositions comprising same, and the methods of treating cancers involving aberrant EGFR activity.

### CROSS-REFERENCE TO RELATED APPLICATIONS

This application claims the benefit of and priority to co-pending U.S. Provisional Patent Application No. 63/375,622, filed on Sep. 14, 2022, and U.S. Provisional Patent Application No. 63/507,876, filed on Jun. 13, 2023, the contents of which are incorporated by reference herein in their entireties.

### STATEMENT REGARDING FEDERALLY SPONSORED RESEARCH OR DEVELOPMENT

This invention was made with government support under grant number R21 GM144753 awarded by the NIH. The government has certain rights in the invention. (37 CFR 401.14 f (4)”

### BACKGROUND

Lung cancer remains the leading cause of cancer-related deaths worldwide, with an estimated 2.20 million new cases and 1.79 million deaths per year.¹ Epidermal growth factor receptor (EGFR, erbB1, HER-1) is an attractive molecular target in lung cancer drug discovery. Activating mutations, which are detected in 10 to 30% of patients with non-small cell lung cancer (NSCLC) such as the point mutation L858R on exon 21 and in-frame exon 19 deletions delE746_A750, confer sensitivity to reversible first-generation EGFR-targeted tyrosine kinase inhibitors (TKIs) such as gefitinib (1) (FIGS. 1-2).²⁻⁵ Unfortunately in NSCLC, up to 40% of patients develop brain metastases (BM) and this number is likely to increase as treatment options continue to improve life expectancy for patients with advanced disease.⁶ As such, BM are a significant risk and result in a poor prognosis for NSCLC patients being treated with poorly central nervous system (CNS) penetrant TKIs.⁶⁻¹⁰ Multiple studies have revealed the low CNS penetrability of a majority of marketed EGFR TKIs due to active efflux by transporters such as P-glycoprotein (P-gp) and breast cancer resistance protein (BCRP), which are highly enriched at the blood-brain barrier (BBB), and in combination with tight junctions, ultimately exclude up to 98% of all drugs from the CNS (FIG. 1).⁷⁻¹⁰ Thus, future treatment options for NSCLC would benefit by improved CNS disposition thereby enabling treatment of the local disease and of ensuing BMs.

### SUMMARY

In accordance with the purpose(s) of the present disclosure, as embodied and broadly described herein, the disclosure, in one aspect, relates to scaffold molecules that inhibit epidermal growth factor receptor (EGFR), methods of making same, pharmaceutical compositions comprising same, and methods of treating cancers involving aberrant EGFR activity.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows the chemical structures of select approved EGFR tyrosine kinase inhibitors (TKIs) and their efflux status against efflux transporters.

FIG. 2 shows the novel bioisosteric modification.

FIG. 3 shows an alternative synthetic route towards hydroxylamine precursor (17).

FIG. 4 shows the synthesis of hydroxylamine-based EGFR inhibitor (6).

FIGS. 5 and 6 show exemplary procedures for making the compounds described herein.

FIG. 7 shows hydroxylamine-based EGFR inhibitors described herein.

FIG. 8 shows the synthesis of hydroxylamine-based EGFR inhibitor (15)

TABLE 1 shows in vitro ADMET properties of gefitinib and the hydroxylamine-based EGFR inhibitors. Values represent the mean of n=2 independent replicates unless otherwise states. Negative ames result on 15 valid up to 50 uM, after which bacterial cytotoxicity was observed. Aq. Sol., aqueous solubility; LMClᵢₙₜ, intrinsic clearance in liver microsomes; HEPClᵢₙₜ, intrinsic clearance in hepatocytes; t_{1/2}, half-life; Pₐₚₚ, apparent permeability; MDCK, Madine-Darby-canine-kidney; MDR1, multidrug resistance 1 (or P-glycoprotein). Abbreviations: H, human; R, rat; nd, not determined.

FIG. 9 shows in vitro antiproliferative activity of gefitinib and the hydroxylamine-based EGFR inhibitors. Hydroxylamine-based EGFR inhibitor (15) displays potent antiproliferative activity in patient-derived non-small-cell lung cancer cell lines HCC827 (EGFR mutation=exon19del), NCI-H3255 (EGFR mutation=L858R) and osimertinib resistant engineered cell lines (Ba/F3-L858R/C797S; Ba/F3 del E746_A750/C797S) while displaying no activity in NCI-H1975 (EGFR mutation=L858R/T790M) and minimal activity in A431 cell line (overexpressed EGFR^{wt}) (72 h dosing period). For all antiproliferative assays, points indicate the mean, and error bars indicated SD; n=3 independent replicates; IC₅₀ values (nM) are reported beside the dose-response curves and represent the mean±SEM.

TABLE. 2 shows the additional in vitro ADMET profile of 15. Values represent the mean of n=≥2 independent replicates. In vitro micronucleus negative test results valid to 31 μM+rat liver S9 and 8 μM−rat liver S9, after which cytotoxicity was observed. Aq. Sol., aqueous solubility; f_{u,plasma}%, percent fraction unbound in plasma; f_{u,brain}%, percent fraction unbound in brain; HEPClᵢₙₜ, intrinsic clearance in hepatocytes. Abbreviations: H, human; R, rat; C, cynomolgous monkey; D, dog.

FIG. 10 shows the KINOMEScan non-mutant or lipid kinase screening results of 15 at a screening concentration of 1 μM. The size of circles mapped onto the kinase phylogenetic tree using DiscoverX TREEspot corresponds to strength of binding affinity.

FIG. 11 shows follow up binding (Kd) and biochemical inhibition (IC₅₀) of 15 against kinases that showed <10% activity in KINOMEScan. Results are the mean of n=2 independent replicates.

FIG. 12 shows follow up in vitro antiproliferative activity of 15 in breast cancer cell lines bearing differing HER2 status. 15 displays moderate antiproliferative activity in patient-derived HER2-positive breast cancer cell lines: AU565, SK-BR-3, BT474 and ZR-75-30. For all antiproliferative assays, points indicate the mean, and error bars indicate SD; n=3 independent replicates; IC₅₀ values (nM) are reported beside the dose-response curve and represent the mean±SEM.

FIG. 13 shows the total plasma vs time profile (0 to 24 h) of 15 after administration into SD rats at a single dose of 2 mg/kg IV and 20 mg/kg PO and BALB/c nude mice at 3 mg/kg IV and 30 mg/kg PO. AUC_{0-inf} (nM-h), area under concentration time curve from 0 to ∞; t_{1/2} (h), mean elimination half-life obtained from either intravenous infusion (IV) or oral gavage (PO); F (%), bioavailability (%); Tₘₐₓ (h), time to reach peak plasma concentration; Cₘₐₓ (nM), peak plasma concentration; CL (mL·min⁻¹·kg), clearance obtained from intravenous infusion. For pharmacokinetic profiles, points indicate the mean and error bars indicate SD; n=3 animals per route (n=6 total). Values represent the mean±SD.

FIG. 14 shows the central nervous system pharmacokinetic profile of hydroxylamine-based EGFR inhibitor (15). Total plasma and brain concentration vs time profile (0 to 24 h) of 15 after administration into SD rats at a single dose of 20 mg/kg and CD1 mice at 40 mg/kg by oral gavage (PO) shows excellent brain penetration. Kₚ brain (AUC_{inf}) refers to the brain-to-plasma partitioning coefficient. K_{p,uu} brain (AUC_{inf}) refers to the unbound brain-to-unbound plasma partitioning coefficient. For pharmacokinetic profiles, points indicate the mean and error bars indicate SD; n=3 animals at each time point (n=21 total).

FIG. 15 shows the bioluminescence images of vehicle control and 15 (10 mg/kg PO b.i.d) dosing groups indicating a change in tumor volume over 21 days of treatment.

FIG. 16 shows the plot of intracranial bioluminescence over time of 15 and mean body weight of mice during the study. For bioluminescence plot, points indicate the mean and error bars indicate the SEM; P=0.0059; n=10 mice per group. PDX, patient-derived xenograft. p value was obtained from an unpaired two-tailed t-test comparing the means of vehicle control and 15 (10 mg/kg PO b.i.d) study arms after 21 days of treatment. *P<0.05; **P<0.01. For mean body weight plot, points indicate the mean and error bars indicate the SEM; P=0.2275; n=10 mice per group. P value was obtained from an unpaired two-tailed t-test comparing the means of vehicle control and 15 (10 mg/kg PO b.i.d) study arms after 21 days of treatment. ns, not significant.

FIGS. 17-18 shows exemplary routes towards hydroxylamine-based nitrogen heterocycles.

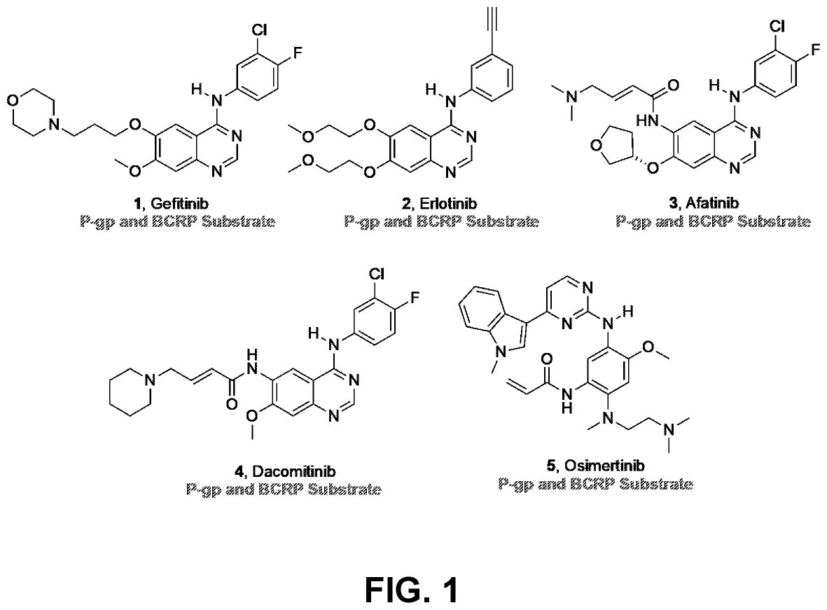

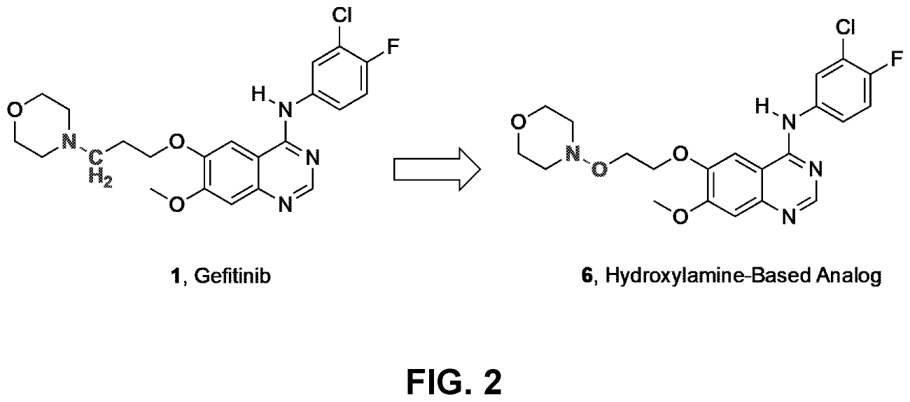

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “an excipient” include, but are not limited to, mixtures or combinations of two or more such excipients, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range. Thus, for example, if a component is in an amount of about 1%, 2%, 3%, 4%, or 5%, where any value can be a lower and upper endpoint of a range, then any range is contemplated between 1% and 5% (e.g., 1% to 3%, 2% to 4%, etc.).

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, “IC₅₀,” is intended to refer to the concentration of a substance (e.g., a compound or a drug) that is required for 50% inhibition of a biological process, or component of a process. For example, IC₅₀ refers to the half maximal (50%) inhibitory concentration (IC) of a substance as determined in a suitable assay.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

The position of a substituent can be defined relative to the positions of other substituents in an aromatic ring. For example, as shown below in relationship to the “R” group, a second substituent can be “ortho,” “para,” or “meta” to the R group, meaning that the second substituent is bonded to a carbon labeled ortho, para, or meta as indicated below. Combinations of ortho, para, and meta substituents relative to a given group or substituent are also envisioned and should be considered to be disclosed.

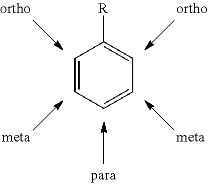

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “alkanediyl” as used herein, refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂— (methylene), —CH₂CH₂—, —CH₂C(CH₃)₂CH₂—, and —CH₂CH₂CH₂— are non-limiting examples of alkanediyl groups.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cyclooctynyl, cyclononynyl, and the like. The term “heterocycloalkynyl” is a type of cycloalkenyl group as defined above and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl to two aryl groups that are bound together via a fused ring structure, as in naphthalene, or are attached via one or more carbon-carbon bonds, as in biphenyl. Fused aryl groups including, but not limited to, indene and naphthalene groups are also contemplated.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) and —N(-alkyl)₂, where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein.

The terms “halo,” “halogen” or “halide,” as used herein can be used interchangeably and refer to F, Cl, Br, or I.

The terms “pseudohalide,” “pseudohalogen” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxy” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” . . . “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{•} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of Rt, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, sulfur, fluorine and chlorine, such as ²H, ³H, ¹³C, ¹⁴C, ¹⁵N, ¹⁸O, ¹⁷O, ³⁵S, ¹⁸F, and ³⁶Cl, respectively. Compounds further comprise prodrugs thereof and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

It is also appreciated that certain compounds described herein can be present as an equilibrium of tautomers. For example, ketones with an α-hydrogen can exist in an equilibrium of the keto form and the enol form.

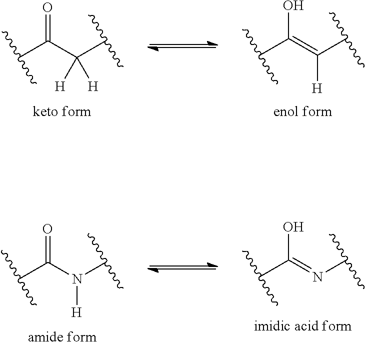 [SMILES: *CC(*)=O ; */C=C(/*)O ; *NC(*)=O ; */N=C(/*)O]

Likewise, amides with an N-hydrogen can exist in an equilibrium of the amide form and the imidic acid form. Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

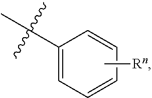

which is understood to be equivalent to a formula:

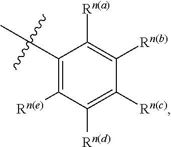

wherein n is typically an integer. That is, Rⁿ is understood to represent five independent substituents, R^{n(a)}, R^{n(b)}, R^{n(c)}, R^{n(d)}, and R^{n(e)}. By “independent substituents,” it is meant that each R substituent can be independently defined. For example, if in one instance R^{n(a)} is halogen, then R^{n(b)} is not necessarily halogen in that instance.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravitreal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as a hematological malignancy, breast cancer, and/or another solid malignancy. The effect can be therapeutic in terms of a partial or complete cure of a disease, condition, symptom or adverse effect attributed to the disease, disorder, or condition. The term “treatment” as used herein can include any treatment of a hematological malignancy, breast cancer, and/or another solid tumor in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “therapeutic” can refer to treating, healing, and/or ameliorating a disease, disorder, condition, or side effect, or to decreasing in the rate of advancement of a disease, disorder, condition, or side effect.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B—F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

### Compounds and Methods of Making and Using the Compounds

In one aspect, disclosed herein is a compound having a structure according to structure I or the pharmaceutically acceptable salt thereof

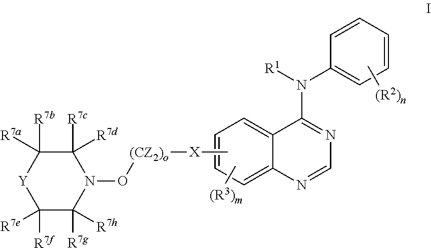

wherein
R¹ is hydrogen, a substituted or unsubstituted linear or branched alkyl group, a substituted or unsubstituted cycloalkyl group or heterocycloalkyl group, or a substituted or unsubstituted aryl group;
n is an integer from 1 to 5, where each R² is independently hydrogen, a substituted or unsubstituted linear or branched alkyl group, a substituted or unsubstituted cycloalkyl group or heterocycloalkyl group, a substituted or unsubstituted aryl group, a halide, or an alkoxy group;
m is an integer from 1 to 3, where each R³ is independently hydrogen, a substituted or unsubstituted linear or branched alkyl group, a substituted or unsubstituted cycloalkyl group or heterocycloalkyl group, a substituted or unsubstituted aryl group, a halide, or an alkoxy group;
is an integer from 1 to 10;
X is O or NR⁴, wherein R⁴ is hydrogen, a substituted or unsubstituted linear or branched alkyl group, a substituted or unsubstituted cycloalkyl group or heterocycloalkyl group, or a substituted or unsubstituted aryl group;
Y is O, NR⁵, or CR^{6a}R^{6b}, wherein R⁵ is hydrogen, a substituted or unsubstituted linear or branched alkyl group, a substituted or unsubstituted cycloalkyl group or heterocycloalkyl group, or a substituted or unsubstituted aryl group, and
R^{6a} and R^{6b} are independently hydrogen, deuterium, a substituted or unsubstituted linear or branched alkyl group, a substituted or unsubstituted cycloalkyl group or heterocycloalkyl group, a substituted or unsubstituted aryl group, or a substituted or unsubstituted amino group;
R^{7a}, R^{7b}, R^{7c}, R^{7d}, R^{7e}, R^{7f}, R^{7g}, and R^{7h} are independently hydrogen, deuterium, or a substituted or unsubstituted alkyl group; and
each Z is independently hydrogen or deuterium.

In one aspect, X is O in structure I. In another aspect, Y is O in structure I. In another aspect, Y is NR⁵ in structure I, where R⁵ is a C1 to C5 alkyl group. In another aspect, Y is CR^{6a}R^{6b} in structure I, where R^{6a} is hydrogen and R^{6b} is a substituted or unsubstituted amino group. In another aspect, R¹ is hydrogen in structure I. In another aspect, R³ is an alkoxy group in structure 1. In another aspect, R³ is an alkoxy group and m is 1 in structure I. In another aspect, o is an integer from 1 to 5 in structure I. In another aspect, R² is a halide and n is 2 in structure I. In another aspect, R² is fluoride at the ortho position in structure I. In another aspect, R^{7a}, R^{7b}, R^{7c}, R^{7d}, R^{7e}, R^{7f}, R^{7g}, and R^{7h} are each hydrogen in structure I. In another aspect, R^{7a}, R^{7b}, R^{7c}, R^{7d}, R^{7e}, R^{7f}, R^{7g}, and R^{7h} are each deuterium in structure I.

In another aspect, the compound has the structure II or the pharmaceutically acceptable salt thereof

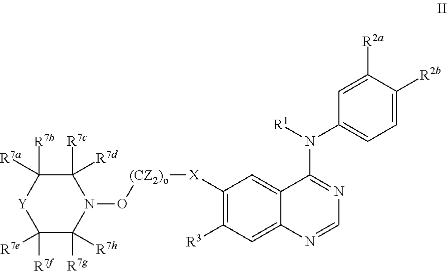

wherein
R¹ is hydrogen, a substituted or unsubstituted linear or branched alkyl group, a substituted or unsubstituted cycloalkyl group or heterocycloalkyl group, or a substituted or unsubstituted aryl group;
R^{2a} and R^{2b} are a halide;
R³ is an alkoxy group;
is an integer from 1 to 5;
X is O or NR⁴, wherein R⁴ is hydrogen, a substituted or unsubstituted linear or branched alkyl group, a substituted or unsubstituted cycloalkyl group or heterocycloalkyl group, or a substituted or unsubstituted aryl group;
Y is O, NR⁵, or CR^{6a}R^{6b}, wherein R⁵ is hydrogen, a substituted or unsubstituted linear or branched alkyl group, a substituted or unsubstituted cycloalkyl group or heterocycloalkyl group, or a substituted or unsubstituted aryl group, and
R^{6a} and R^{6b} are independently hydrogen, deuterium, a substituted or unsubstituted linear or branched alkyl group, a substituted or unsubstituted cycloalkyl group or heterocycloalkyl group, a substituted or unsubstituted aryl group, or a substituted or unsubstituted amino group;
R^{7a}, R^{7b}, R^{7c}, R^{7d}, R^{7e}, R^{7f}, R^{7g}, and R^{7h} are independently hydrogen, deuterium, or a substituted or unsubstituted alkyl group; and
each Z is independently hydrogen or deuterium.

In one aspect, X is O in structure II. In another aspect, Y is O in structure II. In another aspect, Y is NR⁵ in structure II, where R⁵ is a C1 to C5 alkyl group. In another aspect, Y is CR^{6a}R^{6b} in structure II, where R^{6a} is hydrogen and R^{6b} is a substituted or unsubstituted amino group. In another aspect, R¹ is hydrogen in structure II. In another aspect, R³ is a C1 to C10 substituted or unsubstituted linear or branched alkoxy group in structure II. In another aspect, R³ is a methoxy group in structure II. In another aspect, o is an integer from 1 to 5 in structure II. In another aspect, R² is a halide in structure II. In another aspect, R^{2a} is chloride and R^{2b} is fluoride in structure II. In another aspect, R^{2a} is fluoride and R^{2b} is chloride in structure II. In another aspect, R^{7a}, R^{7b}, R^{7c}, R^{7d}, R^{7e}, R^{7f}, R^{7g}, and R^{7h} are each hydrogen in structure II. In another aspect, R^{7a}, R^{7b}, R^{7c}, R^{7d}, R^{7e}, R^{7f}, R^{7g}, and R^{7h} are each deuterium in structure II.

In another aspect, the compound has the structure III or the pharmaceutically acceptable salt thereof

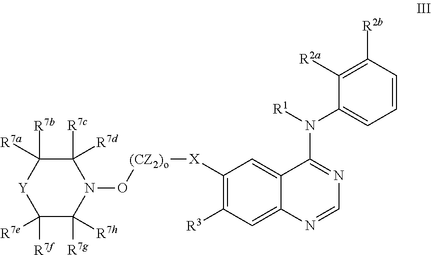

wherein
R¹ is hydrogen, a substituted or unsubstituted linear or branched alkyl group, a substituted or unsubstituted cycloalkyl group or heterocycloalkyl group, or a substituted or unsubstituted aryl group;
R^{2a} and R^{2b} are a halide;
R³ is an alkoxy group;
o is an integer from 1 to 5;
X is O or NR⁴, wherein R⁴ is hydrogen, a substituted or unsubstituted linear or branched alkyl group, a substituted or unsubstituted cycloalkyl group or heterocycloalkyl group, or a substituted or unsubstituted aryl group;
Y is O, NR⁵, or CR^{6a}R^{6b}, wherein R⁵ is hydrogen, deuterium, a substituted or unsubstituted linear or branched alkyl group, a substituted or unsubstituted cycloalkyl group or heterocycloalkyl group, or a substituted or unsubstituted aryl group, and
R^{6a} and R^{6b} are independently hydrogen, a substituted or unsubstituted linear or branched alkyl group, a substituted or unsubstituted cycloalkyl group or heterocycloalkyl group, a substituted or unsubstituted aryl group, or a substituted or unsubstituted amino group;
R^{7a}, R^{7b}, R^{7c}, R^{7d}, R^{7e}, R^{7f}, R^{7g}, and R^{7h} are independently hydrogen, deuterium, or a substituted or unsubstituted alkyl group; and
each Z is independently hydrogen or deuterium.

In one aspect, X is O in structure III. In another aspect, Y is O in structure III. In another aspect, Y is NR⁵ in structure III, where R⁵ is a C1 to C5 alkyl group. In another aspect, Y is CR^{6a}R^{6b} in structure III, where R^{6a} is hydrogen and R^{6b} is a substituted or unsubstituted amino group. In another aspect, R¹ is hydrogen in structure III. In another aspect, R³ is a C1 to C10 substituted or unsubstituted linear or branched alkoxy group in structure III. In another aspect, R³ is a methoxy group in structure III. In another aspect, o is an integer from 1 to 5 in structure Ill. In another aspect, R² is a halide in structure III. In another aspect, R^{2a} is chloride and R^{2b} is fluoride in structure III. In another aspect, R^{2a} is fluoride and R^{2b} is chloride in structure III. In another aspect, R^{2a} is fluoride and R^{2b} is chloride in structure II. In another aspect, R^{7a}, R^{7b}, R^{7c}, R^{7d}, R^{7e}, R^{7f}, R^{7g}, and R^{7h} are each hydrogen in structure III. In another aspect, R^{7a}, R^{7b}, R^{7c}, R^{7d}, R^{7e}, R^{7f}, R^{7g}, and R^{7h} are each deuterium in structure III.

In another aspect, the compound has the following structure

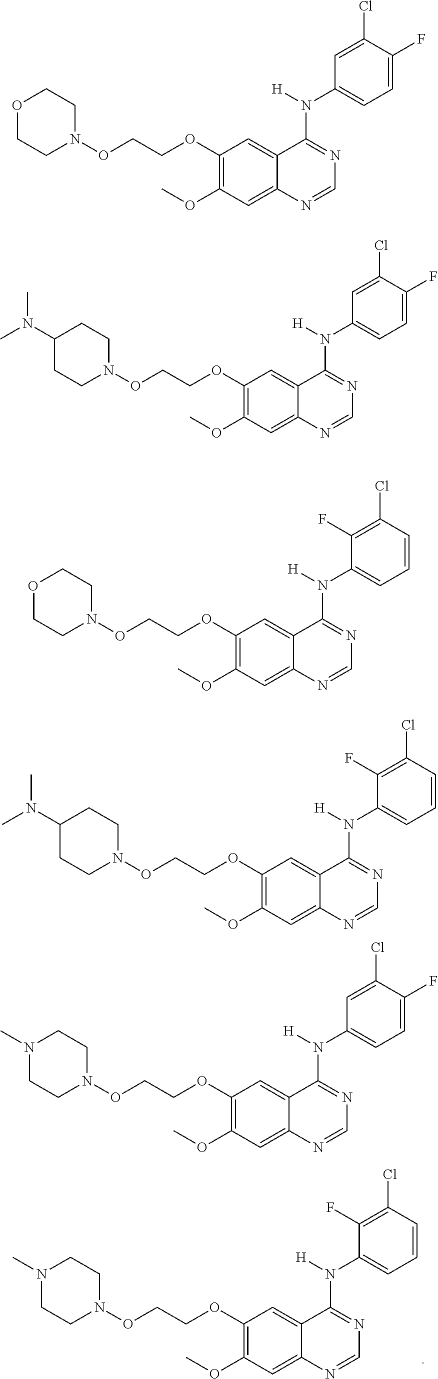

### General Synthetic Method

In one aspect, the compounds described herein can be produced by reacting the compound having the structure IV with the compound having the structure V in the presence of a base

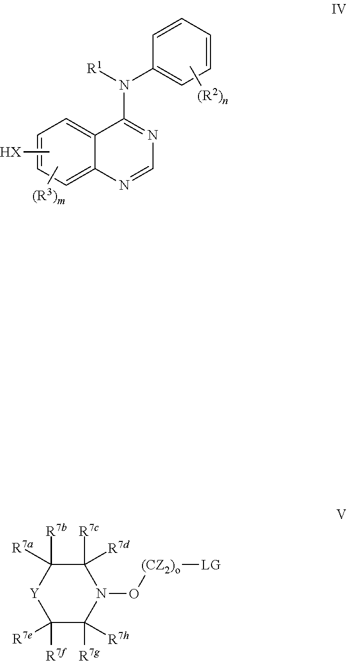

wherein the variables in structures IV and V are as defined above and LG is a leaving group.

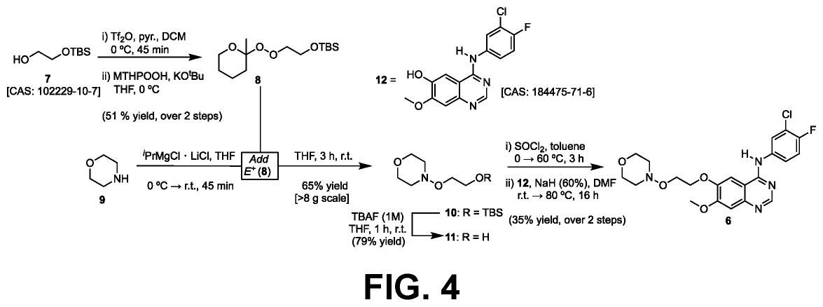

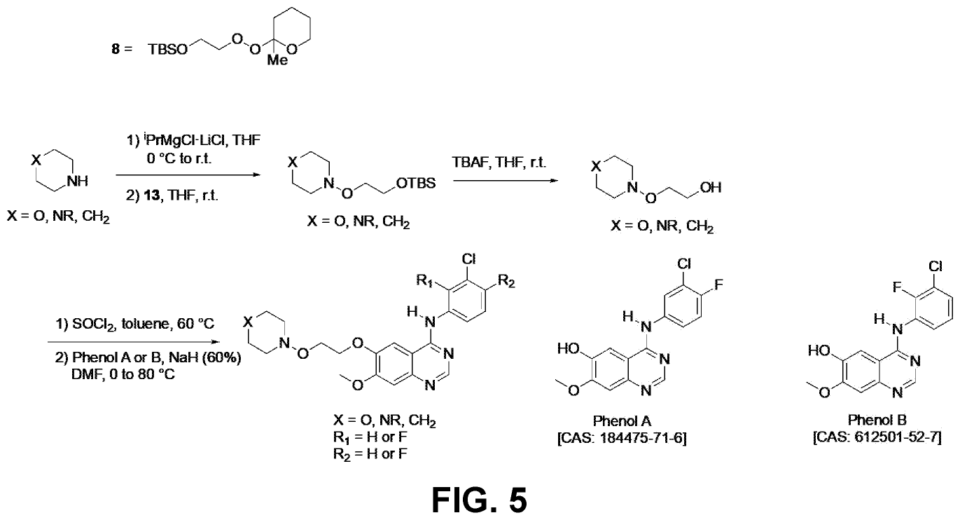

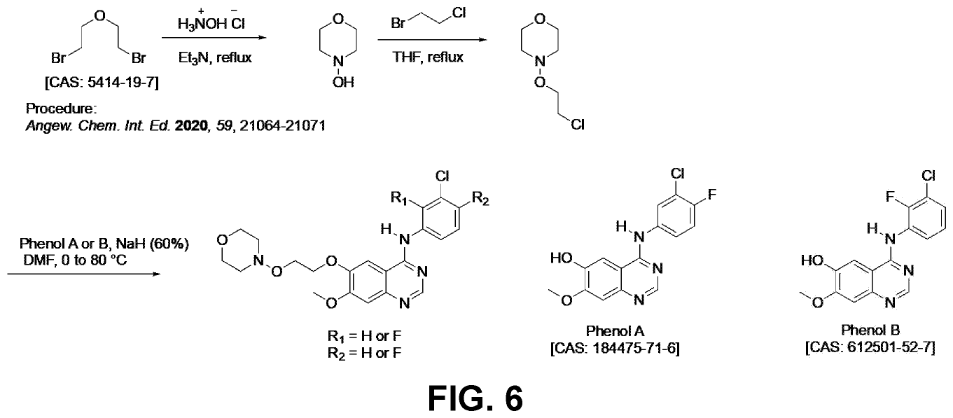

The reaction between the compounds having the structures IV and V are generally performed in an organic solvent, where a suitable amount of base is provided to deprotonate the XH proton in structure IV. In one aspect, the base comprises a hydride, alkoxide, a Grignard reagent, or alkyllithium compound. In one aspect, the leaving group LG in structure V is a halide or a sulfonate group. In one aspect, the compounds described herein can be produced using the procedures provided in FIGS. 4-6 and FIG. 8. The compounds having the structures IV and V can be synthesized using organic techniques or can be purchased. In one aspect, the compound having the structure IV is CAS NO. 184475-71-6.

Also described herein are synthetic methods for producing piperazinyl and morpholino hydroxylamines in large scale and high yield. The synthetic approach requires a minimal number of steps and can produce piperazinyl and morpholino hydroxylamines on a multi-gram scale. The piperazinyl and morpholino hydroxylamines are useful intermediates for producing the compounds described herein.

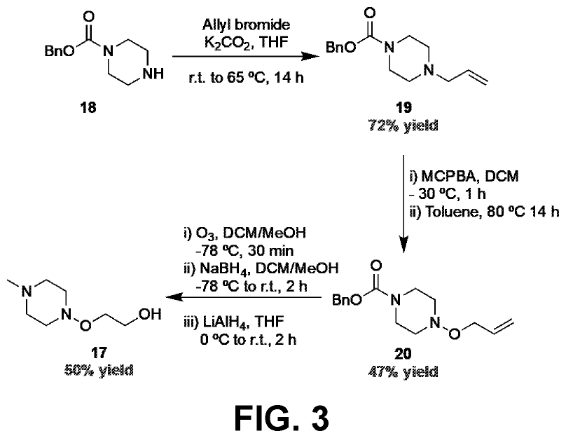

FIG. 3 and FIGS. 17-18 provide exemplary general synthetic procedures for producing the piperazinyl and morpholino hydroxylamines.

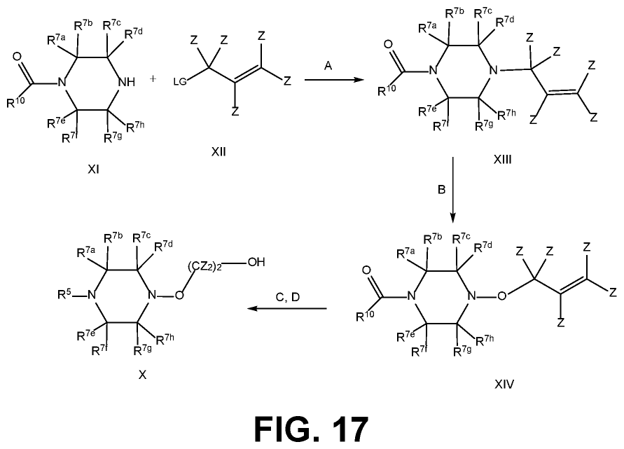

Referring to FIG. 17, four steps A-D are used to produce piperazinyl hydroxylamines having the structure X

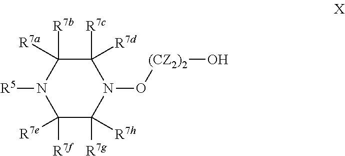

wherein
   R⁵ is a substituted or unsubstituted linear or branched alkyl group, and
   R^{7a}, R^{7b}, R^{7c}, R^{7d}, R^{7e}, R^{7f}, R^{7g}, and R^{7h} are independently hydrogen, deuterium, or a substituted or unsubstituted alkyl group; and
each Z is independently hydrogen or deuterium,

In one aspect, the method for making the piperazinyl hydroxylamines having the structure X involves
(A) reacting a compound having the structure XI with a compound having the structure XII in the presence of a base to produce a compound having the structure XIII

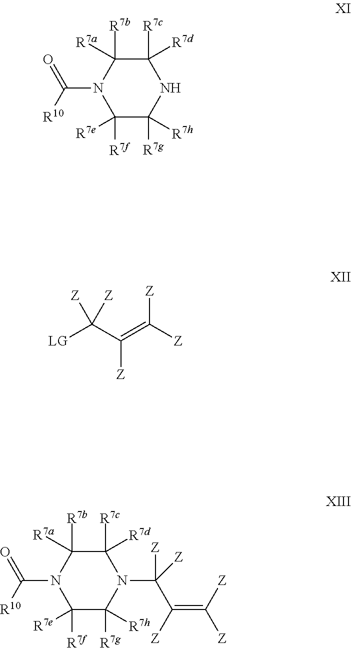

wherein R¹⁰ is a substituted or unsubstituted linear or branched alkyl group, or a substituted or unsubstituted linear or branched alkoxy group, and
LG is leaving group;
(B) reacting a compound having the structure XIII with a first oxidizing agent with heating to produce a compound having the structure XIV

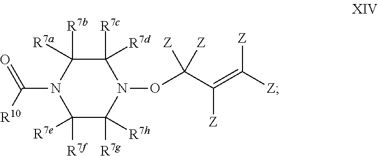

(C) reacting a compound having the structure XIV with (i) a second oxidizing agent followed by (ii) a first reducing agent to produce a first intermediate; and
(D) reacting the first intermediate with a second reducing agent to produce a compound having the structure X.

Step A involves reacting a compound having the structure XI with a compound having the structure XII in the presence of a base to produce a compound having the structure XIII

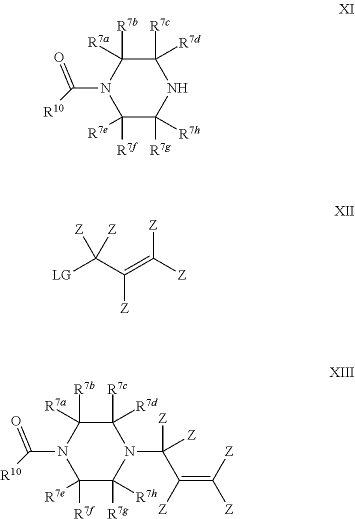

wherein
   R⁵ is a substituted or unsubstituted linear or branched alkyl group, and
   R^{7a}, R^{7b}, R^{7c}, R^{7d}, R^{7e}, R^{7f}, R^{7g}, and R^{7h} are independently hydrogen, deuterium, or a substituted or unsubstituted alkyl group;
each Z is independently hydrogen or deuterium;
   R¹⁰ is a substituted or unsubstituted linear or branched alkyl group, or a substituted or unsubstituted linear or branched alkoxy group, and
   LG is leaving group;

In one aspect, R¹⁰ in structures XI and XIII is a C1-C5 linear or branched alkyl group. In another aspect, R¹⁰ in structures XI and XIII is a C1-C5 linear or branched alkoxy group. In another aspect, R¹⁰ in structures XI and XIII is a C1-C5 linear or branched alkoxy group substituted with an aryl group. In another aspect, R¹⁰ in structures XI and XIII is a benzyloxy group.

Step A involves allylation of the compound having the structure XI with the allyl compound having the structure XII in the presence of a base. In one aspect, leaving group LG of the allyl compound XII is a halide, sulfonate, carbonate, or phosphate. In another aspect, the leaving group is bromide. In one aspect, the allyl compound XII can be partially or completely deuterated. In another aspect, each Z in allyl compound XII is hydrogen. The molar ratio of the compound having the structure XI to the allyl compound having the structure XII can be from 0.5:1 to 1:3, or 0.5:1, 1:1, 1:1.5, 1:2, 1:2.3, or 1:3, where any value can be a lower or upper endpoint of a range (e.g., 1:1.5 to 1:2).

Step A is conducted in an organic solvent. In one aspect, the organic solvent is an aprotic organic solvent such as, for example, tetrahydrofuran (THF), 2-methyltetrahydrofuran (2-methyl THF), diethyl ether, methyl tert-butyl ether (MTBE), 1,4-dioxane, 1,2-dimethoxyethane, pentane, hexanes, heptanes, cyclohexanes, N,N′-dimethylpropyleneurea (DMPU), or a combination thereof.

The base is a compound that can deprotonate the amino proton on the piperazine ring of compound XI. In one aspect, the comprises a carbonate, hydroxide, phosphate, hydride, dialkylamide, or hexamethyldisilazide. The molar ratio of the compound having the structure XI to the base can be from 0.5:1 to 1:3, or 0.5:1, 1:1, 1:1.5, 1:2, 1:2.3, or 1:3, where any value can be a lower or upper endpoint of a range (e.g., 1:1.5 to 1:2).

In one aspect, step A is conducted at elevated temperature. In one aspect, the reaction in step A is conducted at a temperature of from about 25° C. to about 100° C., or 25° C., 35° C., 45° C., 55° C., 65° C., 75° C., 85° C., 95° C., or 100° C., where any value can be a lower or upper endpoint of a range (e.g., 55° C. to 75° C.). After step A, a compound having the structure XIV is produced. The compound having the structure XIII can be subsequently purified using techniques known in the art.

Step B involves oxidation of the compound having the structure XIII followed by Meisenheimer rearrangement to produce a compound having the structure XIV.

In one aspect, step B comprises the steps of
(i) reacting a compound having the structure XIII with a first oxidizing agent in a first organic solvent to produce a first composition;
(ii) adding an aqueous base to the first composition to produce a second composition comprising an organic layer and an aqueous layer;
(iii) separating the organic layer from the aqueous layer;
(iv) removing the first organic solvent from the organic layer to produce a residue;
(v) dissolving the residue in a second organic solvent to produce a second composition; and
(vi) heating the second composition from about 50° C. to about 100° C. to produce the compound having the structure XIV.

In one aspect, the first oxidizing agent in step B comprises a peroxyacid, oxone, or hydrogen peroxide/acetic acid. In another aspect, the first oxidizing agent in step B comprises meta-chloroperoxybenzoic acid. In another aspect, the molar ratio of the first oxidizing agent to the compound having the structure XIII is from 0.95:1 to 1:1.05.

Step B is conducted in an organic solvent, where the solvent does not react with the first oxidant. In one aspect, the first organic solvent is dichloromethane. The first solvent can then be removed using techniques known in the art, the remaining residue is then dissolved in a second organic solvent to produce a second composition. In one aspect, second organic solvent is a higher boiling solvent such as, for example toluene. In one aspect, the second composition in step B is conducted at a temperature of from about 50° C. to about 100° C., or 50° C., 60° C., 70° C., 80° C., 90° C., or 100° C., where any value can be a lower or upper endpoint of a range (e.g., 70° C. to 90° C.). During the heating step, Meisenheimer rearrangement occurs to produce a compound having the structure XIV. The compound having the structure XIIV can be subsequently purified using techniques known in the art.

Step C involves oxidation of the alkenyl group in the compound having the structure XIIV followed by reduction to produce a first intermediate.

In one aspect, step C comprises the steps of
(i) reacting a compound having the structure XIV with a second oxidizing agent in a third organic solvent to produce a third composition; and
(ii) mixing the first reducing agent with the third composition to produce the first intermediate.

In one aspect, the second oxidizing agent used in step C comprises ozone or osmium tetroxide with sodium metaperiodate. Step C is conducted in an organic solvent. In one aspect, third organic solvent comprises an alcohol (e.g., methanol, ethanol, butanol, or any combination thereof) and an aprotic solvent (e.g., tetrahydrofuran (THF), 2-methyltetrahydrofuran (2-methyl THF), diethyl ether, methyl tert-butyl ether (MTBE), 1,4-dioxane, 1,2-dimethoxyethane, pentane, hexanes, heptanes, cyclohexanes, N,N′-dimethylpropyleneurea (DMPU), or any combination thereof).

In one aspect, the compound having the structure XIV is reacted with the second oxidizing agent at a temperature of from about −50° C. to about −100° C., or −50° C., −60° C., −70° C., −80° C., −90° C., or −100° C., where any value can be a lower or upper endpoint of a range (e.g., −70° C. to −90° C.)

After oxidation of the alkenyl group in the compound having the structure XIIV, a first reducing agent is subsequently added to the reaction to produce a first intermediate, which is a terminal alcohol as provided below

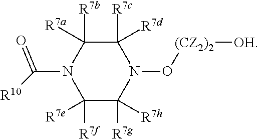

In one aspect, the first reducing agent comprises a hydride, which is any compound that can deliver a hydrogen anion. In one aspect, the first reducing agent comprises a borohydride. In one aspect, the hydride is sodium borohydride, lithium aluminium hydride, or diisobutylaluminium hydride. The molar ratio of the first reducing agent to the compound having the structure XIV is from 1.5:1 to 2.5:1, or 1.5:1, 1.75:1, 2:1, 2.25:1, or 2.5:1, where any value can be a lower or upper endpoint of a range (e.g., 1.75:1 to 2:1). The first intermediate produced in step C can be subsequently purified using techniques known in the art prior to step D.

Step D involves reduction of the carbonyl group in the first intermediate to produce the piperazinyl hydroxylamine compound X.

In one aspect, step D comprises the steps of
(i) dissolving the first intermediate in an aprotic solvent to produce a fourth composition;
(ii) mixing the second reducing agent with the fourth composition to produce the compound having the structure X; and
(iii) isolating and purifying the compound having the structure X.

The first intermediate is dissolved in an aprotic solvent (e.g., tetrahydrofuran (THF), 2-methyltetrahydrofuran (2-methyl THF), diethyl ether, methyl tert-butyl ether (MTBE), 1,4-dioxane, 1,2-dimethoxyethane, pentane, hexanes, heptanes, cyclohexanes, N,N′-dimethylpropyleneurea (DMPU), or any combination thereof) followed by the addition the second reducing agent. In one aspect, the hydride is sodium borohydride, lithium aluminium hydride, or diisobutylaluminium hydride. The molar ratio of the first reducing agent to the compound having the structure XIV is from 2:1 to 4:1, or 2:1, 2.5:1, 3:1, 3.5:1, or 4:1, where any value can be a lower or upper endpoint of a range (e.g., 2.5:1 to 3.5:1).

In one aspect, the first intermediate is reacted with the second reducing agent at a temperature of from about 10° C. to about −50° C., or 10° C., 0° C., −10° C., −20° C., −30° C., −40° C., or −50° C., where any value can be a lower or upper endpoint of a range (e.g., −10° C. to −30° C.). The piperazinyl hydroxylamine compound X produced in step D can be subsequently purified using techniques known in the art prior to step D.

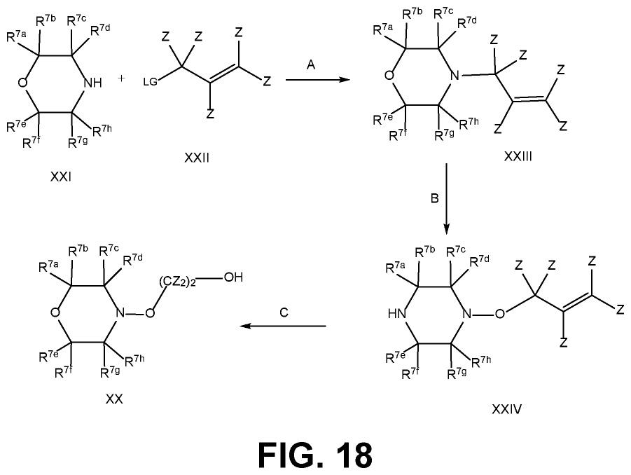

Referring to FIG. 18, three steps A-C are used to produce morpholino hydroxylamines having the structure XX

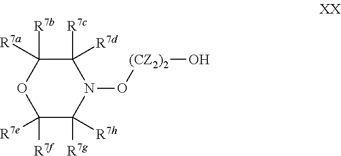

wherein
R^{7a}, R^{7b}, R^{7c}, R^{7d}, R^{7e}, R^{7f}, R^{7g}, and R^{7h} are independently hydrogen, deuterium, or a substituted or unsubstituted alkyl group; and
each Z is independently hydrogen or deuterium,

In one aspect, the method for making the morpholino hydroxylamines having the structure XX involves
(A) reacting a compound having the structure XXI with a compound having the structure XXII in the presence of a base to produce a compound having the structure XXIII

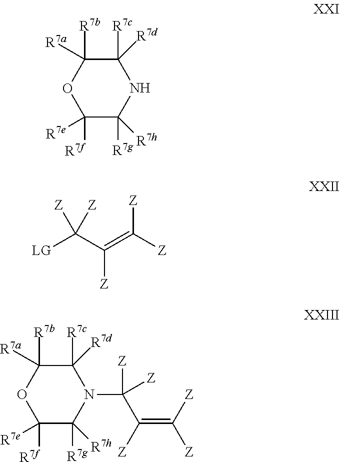

wherein LG is leaving group;
(B) reacting a compound having the structure XXIII with a first oxidizing agent to produce a compound having the structure XXIV

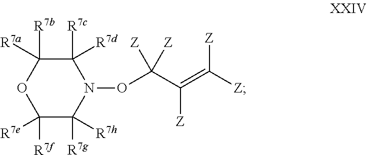

and
(C) reacting the compound having the structure XXIV with (i) a second oxidizing agent followed by (ii) a first reducing agent to produce a compound having the structure XX.

The same reaction conditions and reagents used in steps A-C for producing the piperazinyl hydroxylamines can be used to produce the morpholino hydroxylamines having the structure XX, where step D is not required to produce the morpholino hydroxylamines.

Exemplary methods for producing compounds described herein, as well as characterization information, are provided in the Examples. Solvents, temperatures, presence or absence of protecting groups, and other reaction conditions may vary according to the specific substituents in the compound being synthesized.

Exemplary methods for producing compounds described herein, as well as characterization information, are provided in the Examples. Solvents, temperatures, presence or absence of protecting groups, and other reaction conditions may vary according to the specific substituents in the compound being synthesized.

### Pharmaceutical Compositions

In various aspects, the present disclosure relates to pharmaceutical compositions comprising a therapeutically effective amount of at least one disclosed compound, at least one product of a disclosed method, or a pharmaceutically acceptable salt thereof. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one disclosed compound, at least one product of a disclosed method, or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration orally, nasally, via inhalation, parenterally, paracancerally, transmucosally, transdermally, intramuscularly, intravenously, intradermally, subcutaneously, intraperitoneally, intraventricularly, intracranially and intratumorally.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural and intrasternal injection and infusion.

In various aspects, the present disclosure also relates to a pharmaceutical composition comprising a pharmaceutically acceptable carrier or diluent and, as active ingredient, a therapeutically effective amount of a disclosed compound, a product of a disclosed method of making, a pharmaceutically acceptable salt, a hydrate thereof, a solvate thereof, a polymorph thereof, or a stereochemically isomeric form thereof. In a further aspect, a disclosed compound, a product of a disclosed method of making, a pharmaceutically acceptable salt, a hydrate thereof, a solvate thereof, a polymorph thereof, or a stereochemically isomeric form thereof, or any subgroup or combination thereof may be formulated into various pharmaceutical forms for administration purposes.

In practice, the compounds of the present disclosure, or pharmaceutically acceptable salts thereof, of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

The pharmaceutical compositions disclosed herein comprise a compound of the present disclosure (or pharmaceutically acceptable salts thereof) as an active ingredient, a pharmaceutically acceptable carrier, and optionally one or more additional therapeutic agents. In various aspects, the disclosed pharmaceutical compositions can include a pharmaceutically acceptable carrier and a disclosed compound, or a pharmaceutically acceptable salt thereof. In a further aspect, a disclosed compound, or pharmaceutically acceptable salt thereof, can also be included in a pharmaceutical composition in combination with one or more other therapeutically active compounds. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The compounds described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent the most advantageous oral dosage unit forms in which case solid pharmaceutical carriers are obviously employed. However, other dosage forms may be suitable depending upon clinical population (e.g., age and severity of clinical condition), solubility properties of the specific disclosed compound used, and the like. Accordingly, the disclosed compounds can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₈H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentaerythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols, dimethylacetamide, lactamides, lactates, ethyl carbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example Eudragit® RS), copolymerizates of acrylic and methacrylic acid esters and trimethyl ammonium methacrylate (for example Eudragit® RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed compound that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulfoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N,N-diethyl acetamide, N,N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono- or diglycerides or unsaturated compounds such as those containing oleic acid radicals) with ethylene oxide (for example 40 Mol ethylene oxide per 1 Mol glyceride). Examples of oleotriglycerides are olive oil, peanut oil, castor oil, sesame oil, cottonseed oil, corn oil. See also Dr. H. P. Fiedler “Lexikon der Hillsstoffe für Pharmazie, Kostnetik und angrenzende Gebiete” 1971, pages 191-195.

In various aspects, a liquid dosage form can further comprise preservatives, stabilizers, buffer substances, flavor correcting agents, sweeteners, colorants, antioxidants and complex formers and the like. Complex formers which may be for example be considered are: chelate formers such as ethylene diamine retrascetic acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In order to enhance the solubility and/or the stability of a disclosed compound in a disclosed liquid dosage form, a parenteral injection form, or an intravenous injectable form, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also co-solvents such as alcohols may improve the solubility and/or the stability of the compounds according to the present disclosure in pharmaceutical compositions.

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Pharmaceutical compositions of the present disclosure suitable injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions or suspensions, including saline and buffered media. Parenteral vehicles can include mannitol, normal serum albumin, sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

In addition to the pharmaceutical compositions described herein above, the disclosed compounds can also be formulated as a depot preparation. Such long acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

Pharmaceutical compositions of the present disclosure can be in a form suitable for topical administration. As used herein, the phrase “topical application” means administration onto a biological surface, whereby the biological surface includes, for example, a skin area (e.g., hands, forearms, elbows, legs, face, nails, anus and genital areas) or a mucosal membrane. By selecting the appropriate carrier and optionally other ingredients that can be included in the composition, as is detailed herein below, the compositions of the present invention may be formulated into any form typically employed for topical application. A topical pharmaceutical composition can be in a form of a cream, an ointment, a paste, a gel, a lotion, milk, a suspension, an aerosol, a spray, foam, a dusting powder, a pad, and a patch. Further, the compositions can be in a form suitable for use in transdermal devices. These formulations can be prepared, utilizing a compound of the present disclosure, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emollience). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically liquid or semiliquid preparations in which solid particles, including the active agent, are present in a water or alcohol base. Lotions are typically preferred for treating large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information.

Pastes are semisolid dosage forms in which the bioactive agent is suspended in a suitable base. Depending on the nature of the base, pastes are divided between fatty pastes or those made from a single-phase aqueous gel. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; modified cellulose, such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or aqueous alkanolic, but are typically formulated with high alcohol content which, upon application to the skin of a user, quickly evaporates, driving the active ingredient through the upper skin layers to the site of treatment.

Skin patches typically comprise a backing, to which a reservoir containing the active agent is attached. The reservoir can be, for example, a pad in which the active agent or composition is dispersed or soaked, or a liquid reservoir. Patches typically further include a frontal water permeable adhesive, which adheres and secures the device to the treated region. Silicone rubbers with self-adhesiveness can alternatively be used. In both cases, a protective permeable layer can be used to protect the adhesive side of the patch prior to its use. Skin patches may further comprise a removable cover, which serves for protecting it upon storage.

Examples of patch configuration which can be utilized with the present invention include a single-layer or multi-layer drug-in-adhesive systems which are characterized by the inclusion of the drug directly within the skin-contacting adhesive. In such a transdermal patch design, the adhesive not only serves to affix the patch to the skin, but also serves as the formulation foundation, containing the drug and all the excipients under a single backing film. In the multi-layer drug-in-adhesive patch a membrane is disposed between two distinct drug-in-adhesive layers or multiple drug-in-adhesive layers are incorporated under a single backing film.

Examples of pharmaceutically acceptable carriers that are suitable for pharmaceutical compositions for topical applications include carrier materials that are well-known for use in the cosmetic and medical arts as bases for e.g., emulsions, creams, aqueous solutions, oils, ointments, pastes, gels, lotions, milks, foams, suspensions, aerosols and the like, depending on the final form of the composition. Representative examples of suitable carriers according to the present invention therefore include, without limitation, water, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials commonly employed in cosmetic and medicinal compositions. Other suitable carriers according to the present invention include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

Topical compositions of the present disclosure can, if desired, be presented in a pack or dispenser device, such as an FDA-approved kit, which may contain one or more unit dosage forms containing the active ingredient. The dispenser device may, for example, comprise a tube. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising the topical composition of the invention formulated in a pharmaceutically acceptable carrier may also be prepared, placed in an appropriate container, and labeled for treatment of an indicated condition.

Another patch system configuration which can be used by the present invention is a reservoir transdermal system design which is characterized by the inclusion of a liquid compartment containing a drug solution or suspension separated from the release liner by a semi-permeable membrane and adhesive. The adhesive component of this patch system can either be incorporated as a continuous layer between the membrane and the release liner or in a concentric configuration around the membrane. Yet another patch system configuration which can be utilized by the present invention is a matrix system design which is characterized by the inclusion of a semisolid matrix containing a drug solution or suspension which is in direct contact with the release liner. The component responsible for skin adhesion is incorporated in an overlay and forms a concentric configuration around the semisolid matrix.

Pharmaceutical compositions of the present disclosure can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Pharmaceutical compositions containing a compound of the present disclosure, and/or pharmaceutically acceptable salts thereof, can also be prepared in powder or liquid concentrate form.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed compound formulated in a compatible pharmaceutical carrier may also be prepared, placed in an appropriate container, and labeled for treatment of an indicated condition.

The exact dosage and frequency of administration depends on the particular disclosed compound, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compounds of the present disclosure.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

In one aspect, an appropriate dosage level will generally be about 0.01 to 1000 mg of a compound described herein per kg patient body weight per day and can be administered in single or multiple doses. In various aspects, the dosage level will be about 0.1 to about 500 mg/kg per day, about 0.1 to 250 mg/kg per day, or about 0.5 to 100 mg/kg per day. A suitable dosage level can be about 0.01 to 1000 mg/kg per day, about 0.01 to 500 mg/kg per day, about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the form of tablets containing 1.0 to 1000 mg of the active ingredient, particularly 1.0, 5.0, 10, 15, 20, 25, 50, 75, 100, 150, 200, 250, 300, 400, 500, 600, 750, 800, 900 and 1000 mg of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

Such unit doses as described hereinabove and hereinafter can be administered more than once a day, for example, 2, 3, 4, 5 or 6 times a day. In various aspects, such unit doses can be administered 1 or 2 times per day, so that the total dosage for a 70 kg adult is in the range of 0.001 to about 15 mg per kg weight of subject per administration. In a further aspect, dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The disclosed pharmaceutical compositions can further comprise other therapeutically active compounds, which are usually applied in the treatment of the above mentioned pathological or clinical conditions.

It is understood that the disclosed compositions can be prepared from the disclosed compounds. It is also understood that the disclosed compositions can be employed in the disclosed methods of using.

As already mentioned, the present disclosure relates to a pharmaceutical composition comprising a therapeutically effective amount of a disclosed compound, a product of a disclosed method of making, a pharmaceutically acceptable salt, a hydrate thereof, a solvate thereof, a polymorph thereof, and a pharmaceutically acceptable carrier. Additionally, the present disclosure relates to a process for preparing such a pharmaceutical composition, characterized in that a pharmaceutically acceptable carrier is intimately mixed with a therapeutically effective amount of a compound according to the present disclosure.

### Methods for Treatment of Cancers in Subjects

In one aspect, disclosed herein is a method for the treatment of a cancer in a subject, the method including the step of administering to the subject a therapeutically effective amount of at least one disclosed compound, or a pharmaceutically acceptable salt thereof, or the disclosed pharmaceutical composition. In some aspects, the subject is a human. In another aspect, the subject has been diagnosed with a need for treatment of the cancer prior to the administering step. In some aspects, the method further includes the step of identifying a subject in need of treatment of the cancer. In one aspect, the cancer is selected from non-small cell lung cancer (NSCLC), neuroblastoma, glioblastoma multiforme, metastatic brain cancer, brain cancer, prostate cancer or breast cancer.

In one aspect, the compounds described herein can treat brain metastases (BM). Metastases to the brain remain a significant problem in lung cancer.⁶ Treatment by the majority of small molecule targeted therapies is severely limited by efflux transporters at the blood-brain-barrier (BBB).⁶⁻¹⁰ Unfortunately in NSCLC, up to 40% of patients develop brain metastases (BM) and this number is likely to increase as treatment options continue to improve life expectancy for patients with advanced disease.⁶ As such, BM are a significant risk and result in a poor prognosis for NSCLC patients being treated with poorly central nervous system (CNS) penetrant tyrosine kinase inhibitors (TKIs).⁷⁻¹⁰ As demonstrated herein, the compounds described herein exhibit high brain penetration and potent activity in osimertinib-resistant cell lines.

In another aspect, disclosed herein is a method for inhibiting epidermal growth factor receptor (EGFR) in a subject, including the step of administering to the subject a therapeutically effective amount of at least one disclosed compound, or a pharmaceutically acceptable salt thereof, or a disclosed pharmaceutical composition. In one aspect, the subject is a human.

In one aspect, the compound is administered orally to the subject. In another aspect, the compound is administered at a dosage of from about 50 mg per day to about 1,000 mg per day, or about 50 mg per day, 50 mg per day, 100 mg per day, 150 mg per day, 200 mg per day, 250 mg per day, 300 mg per day, 350 mg per day, 400 mg per day, 450 mg per day, 500 mg per day, 550 mg per day, 600 mg per day, 650 mg per day, 700 mg per day, 750 mg per day, 800 mg per day, 850 mg per day, 900 mg per day, 950 mg per day, or 1,000 mg per day, where any value can be a lower and upper endpoint of a range (e.g., 100 mg per day to 300 mg per day).

As demonstrated in the Examples, the compounds described herein lack mutagenicity and demonstrate comparable metabolic stability to gefitinib. Additionally, the compounds have improved permeability and reduced efflux as measured in cellular assays. The compounds described herein can be used alone or in combination with other chemotherapeutic agents and/or radiation.

### ASPECTS

Aspect 1. A compound having structure I or the pharmaceutically acceptable salt thereof

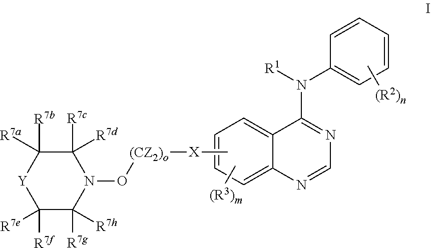

wherein
R¹ is hydrogen, a substituted or unsubstituted linear or branched alkyl group, a substituted or unsubstituted cycloalkyl group or heterocycloalkyl group, or a substituted or unsubstituted aryl group;
n is an integer from 1 to 5, where each R² is independently hydrogen, a substituted or unsubstituted linear or branched alkyl group, a substituted or unsubstituted cycloalkyl group or heterocycloalkyl group, a substituted or unsubstituted aryl group, a halide, or an alkoxy group;
m is an integer from 1 to 3, where each R³ is independently hydrogen, a substituted or unsubstituted linear or branched alkyl group, a substituted or unsubstituted cycloalkyl group or heterocycloalkyl group, a substituted or unsubstituted aryl group, a halide, or an alkoxy group;
o is an integer from 1 to 10;
X is O or NR⁴, wherein R⁴ is hydrogen, a substituted or unsubstituted linear or branched alkyl group, a substituted or unsubstituted cycloalkyl group or heterocycloalkyl group, or a substituted or unsubstituted aryl group;
Y is O, NR⁵, or CR^{6a}R^{6b}, wherein R⁵ is hydrogen, a substituted or unsubstituted linear or branched alkyl group, a substituted or unsubstituted cycloalkyl group or heterocycloalkyl group, or a substituted or unsubstituted aryl group, and
R^{6a} and R^{6b} are independently hydrogen, deuterium, a substituted or unsubstituted linear or branched alkyl group, a substituted or unsubstituted cycloalkyl group or heterocycloalkyl group, a substituted or unsubstituted aryl group, or a substituted or unsubstituted amino group;
R^{7a}, R^{7b}, R^{7c}, R^{7d}, R^{7e}, R^{7f}, R^{7g}, and R^{7h} are independently hydrogen, deuterium, or a substituted or unsubstituted alkyl group; and
each Z is independently hydrogen or deuterium.

Aspect 2. The compound of Aspect 1, wherein X is O.

Aspect 3. The compound of Aspect 1 or 2, wherein Y is O.

Aspect 4. The compound of Aspect 1 or 2, wherein Y is NR⁵, where R⁵ is a C1 to C5 alkyl group.

Aspect 5. The compound of Aspect 1 or 2, wherein Y is CR^{6a}R^{6b}, where R^{6a} is hydrogen and R^{6b} is a substituted or unsubstituted amino group.

Aspect 6. The compound of any one of Aspects 1-5, wherein R¹ is hydrogen.

Aspect 7. The compound of any one of Aspects 1-6, wherein R³ is an alkoxy group.

Aspect 8. The compound of any one of Aspects 1-6, wherein R³ is an alkoxy group and m is 1.

Aspect 9. The compound of any one of Aspects 1-8, wherein o is an integer from 1 to 5.

Aspect 10. The compound of any one of Aspects 1-9, wherein R² is a halide and n is 2.

Aspect 11. The compound of any one of Aspects 1-10, wherein R² is fluoride at the ortho position.

Aspect 12. The compound of Aspect 1, wherein the compound has the structure II or the pharmaceutically acceptable salt thereof

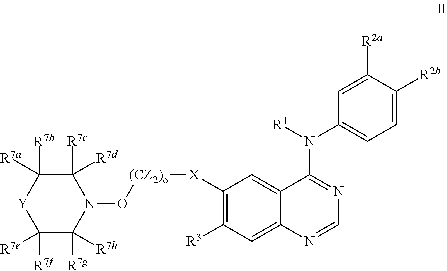

Wherein
R¹ is hydrogen, a substituted or unsubstituted linear or branched alkyl group, a substituted or unsubstituted cycloalkyl group or heterocycloalkyl group, or a substituted or unsubstituted aryl group;
R^{2a} and R^{2b} are a halide;
R³ is an alkoxy group;
o is an integer from 1 to 5;
X is O or NR⁴, wherein R⁴ is hydrogen, a substituted or unsubstituted linear or branched alkyl group, a substituted or unsubstituted cycloalkyl group or heterocycloalkyl group, or a substituted or unsubstituted aryl group;
Y is O, NR⁵, or CR^{6a}R^{6b}, wherein R⁵ is hydrogen, a substituted or unsubstituted linear or branched alkyl group, a substituted or unsubstituted cycloalkyl group or heterocycloalkyl group, or a substituted or unsubstituted aryl group, and
R^{6a} and R^{6b} are independently hydrogen, deuterium, a substituted or unsubstituted linear or branched alkyl group, a substituted or unsubstituted cycloalkyl group or heterocycloalkyl group, a substituted or unsubstituted aryl group, or a substituted or unsubstituted amino group;
R^{7a}, R^{7b}, R^{7c}, R^{7d}, R^{7e}, R^{7f}, R^{7g}, and R^{7h} are independently hydrogen, deuterium, or a substituted or unsubstituted alkyl group; and
each Z is independently hydrogen or deuterium.

Aspect 13. The compound of Aspect 12, wherein X is O.

Aspect 14. The compound of Aspect 12 or 13, wherein Y is O.

Aspect 15. The compound of Aspect 12 or 13, wherein Y is NR⁵, where R⁵ is a C1 to C5 alkyl group.

Aspect 16. The compound of Aspect 12 or 13, wherein Y is CR^{6a}R^{6b}, where R^{6a} is hydrogen and R^{6b} is a substituted or unsubstituted amino group.

Aspect 17. The compound of any one of Aspects 12 to 16, wherein R¹ is hydrogen.

Aspect 18. The compound of any one of Aspects 12 to 17, wherein R³ is a C1 to C10 substituted or unsubstituted linear or branched alkoxy group.

Aspect 19. The compound of any one of Aspects 12 to 17, wherein R³ is a methoxy group.

Aspect 20. The compound of any one of Aspects 12 to 19, wherein o is an integer from 1 to 5.

Aspect 21. The compound of any one of Aspects 12 to 20, wherein R² is a halide.

Aspect 22. The compound of any one of Aspects 12 to 21, wherein R^{2a} is chloride and R^{2b} is fluoride.

Aspect 23. The compound of any one of Aspects 12 to 21, wherein R^{2a} is fluoride and R^{2b} is chloride.

Aspect 24. The compound of Aspect 1, wherein the compound has the structure III or the pharmaceutically acceptable salt thereof

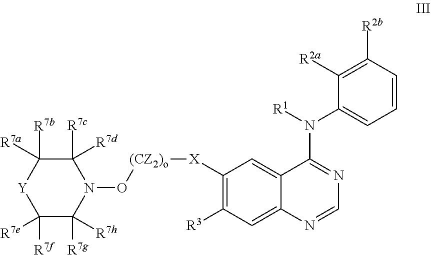

Wherein
R¹ is hydrogen, a substituted or unsubstituted linear or branched alkyl group, a substituted or unsubstituted cycloalkyl group or heterocycloalkyl group, or a substituted or unsubstituted aryl group;
R^{2a} and R^{2b} are a halide;
R³ is an alkoxy group;
is an integer from 1 to 5;
X is O or NR⁴, wherein R⁴ is hydrogen, a substituted or unsubstituted linear or branched alkyl group, a substituted or unsubstituted cycloalkyl group or heterocycloalkyl group, or a substituted or unsubstituted aryl group;
Y is O, NR⁵, or CR^{6a}R^{6b}, wherein R⁵ is hydrogen, deuterium, a substituted or unsubstituted linear or branched alkyl group, a substituted or unsubstituted cycloalkyl group or heterocycloalkyl group, or a substituted or unsubstituted aryl group, and
R^{6a} and R^{6b} are independently hydrogen, a substituted or unsubstituted linear or branched alkyl group, a substituted or unsubstituted cycloalkyl group or heterocycloalkyl group, a substituted or unsubstituted aryl group, or a substituted or unsubstituted amino group;
R^{7a}, R^{7b}, R^{7c}, R^{7d}, R^{7e}, R^{7f}, R^{7g}, and R^{7h} are independently hydrogen, deuterium, or a substituted or unsubstituted alkyl group; and
each Z is independently hydrogen or deuterium.

Aspect 25. The compound of Aspect 24, wherein X is O.

Aspect 26. The compound of Aspect 24 or 25, wherein Y is O.

Aspect 27. The compound of Aspect 24 or 25, wherein Y is NR⁵, where R⁵ is a C1 to C5 alkyl group.

Aspect 28. The compound of Aspect 24 or 25, wherein Y is CR^{6a}R^{6b}, where R^{6a} is hydrogen and R^{6b} is a substituted or unsubstituted amino group.

Aspect 29. The compound of any one of Aspects 24-18, wherein R¹ is hydrogen.

Aspect 30. The compound of any one of Aspects 24 to 29, wherein R³ is a C1 to C10 substituted or unsubstituted linear or branched alkoxy group.

Aspect 31. The compound of any one of Aspects 24 to 29, wherein R³ is a methoxy group.

Aspect 32. The compound of any one of Aspects 24-31, wherein o is an integer from 1 to 5.

Aspect 33. The compound of any one of Aspects 24-32, wherein R² is a halide.

Aspect 34. The compound of any one of Aspects 24 to 32, wherein R^{2a} is chloride and R^{2b} is fluoride.

Aspect 35. The compound of any one of Aspects 24 to 32, wherein R^{2a} is fluoride and R^{2b} is chloride.

Aspect 36. The compound of Aspect 1, wherein the compound is

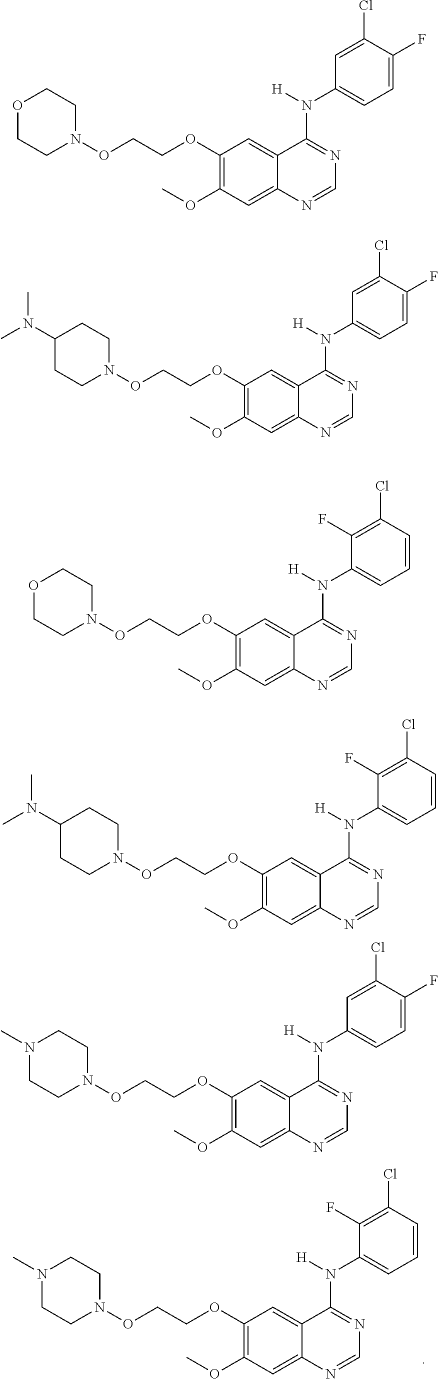

Aspect 37. A pharmaceutical composition comprising the compound of any one of claims 1 to 36 and a pharmaceutically-acceptable carrier.

Aspect 38. A method for treating a subject having non-small cell lung cancer, neuroblastoma, glioblastoma multiforme, metastatic brain cancer, brain cancer, breast cancer, or prostate cancer, the method comprising administering to the subject an effective amount of the compound of any one of Aspects to 36.

Aspect 39. A method for inhibiting epidermal growth factor receptor (EGFR) in a subject, the method comprising administering to the subject an effective amount of the compound in any one of Aspects 1 to 36.

Aspect 40. A method for treating a subject having osimertinib-resistant cancer, the method comprising administering to the subject an effective amount of the compound of any one of claims 1 to 36.

Aspect 41. A method for treating a subject having brain metastases, the method comprising administering to the subject an effective amount of the compound of any one of claims 1 to 36.

Aspect 42. The method of any one of Aspects 38 to 41, wherein the compound is administered orally to the subject.

Aspect 43. The method of any one of Aspects 38 to 42, wherein the compound is administered at a dosage of from about 50 mg per day to about 1,000 mg per day.

Aspect 44. A method for making the compound of any one of Aspects 1 to 16, the method comprising reacting the compound having the structure IV with the compound having the structure V in the presence of a base

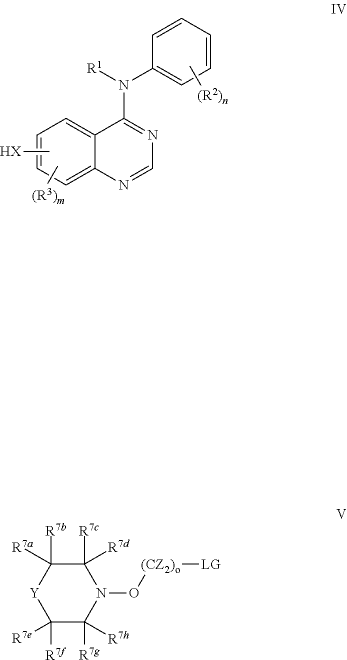

wherein
R¹ is hydrogen, a substituted or unsubstituted linear or branched alkyl group, a substituted or unsubstituted cycloalkyl group or heterocycloalkyl group, or a substituted or unsubstituted aryl group;
R^{2a} and R^{2b} are a halide;
R³ is an alkoxy group;
o is an integer from 1 to 5;
X is O or NR⁴, wherein R⁴ is hydrogen, a substituted or unsubstituted linear or branched alkyl group, a substituted or unsubstituted cycloalkyl group or heterocycloalkyl group, or a substituted or unsubstituted aryl group;
Y is O, NR⁵, or CR^{6a}R^{6b}, wherein R⁵ is hydrogen, a substituted or unsubstituted linear or branched alkyl group, a substituted or unsubstituted cycloalkyl group or heterocycloalkyl group, or a substituted or unsubstituted aryl group, and
R^{6a} and R^{6b} are independently hydrogen, deuterium, a substituted or unsubstituted linear or branched alkyl group, a substituted or unsubstituted cycloalkyl group or heterocycloalkyl group, a substituted or unsubstituted aryl group, or a substituted or unsubstituted amino group;
R^{7a}, R^{7b}, R^{7c}, R^{7d}, R^{7e}, R^{7f}, R^{7g}, and R^{7h} are independently hydrogen, deuterium, or a substituted or unsubstituted alkyl group;
each Z is independently hydrogen or deuterium; and
LG is a leaving group.

Aspect 45. The method of Aspect 44, wherein LG is a halide or a sulfonate group.

Aspect 46. The method of Aspect 44 or 45, wherein the base comprises a hydride, alkoxide, a Grignard reagent, or alkyl lithium compound.

Aspect 47. A method for making a compound having the structure X

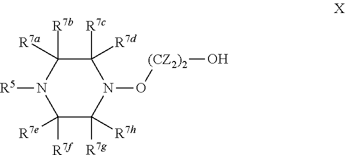

wherein
   R⁵ is a substituted or unsubstituted linear or branched alkyl group, and
   R^{7a}, R^{7b}, R^{7c}, R^{7d}, R^{7e}, R^{7f}, R^{7g}, and R^{7h} are independently hydrogen, deuterium, or a substituted or unsubstituted alkyl group; and
   each Z is independently hydrogen or deuterium,
the method comprising
   (a) reacting a compound having the structure XI with a compound having the structure XII in the presence of a base to produce a compound having the structure XIII

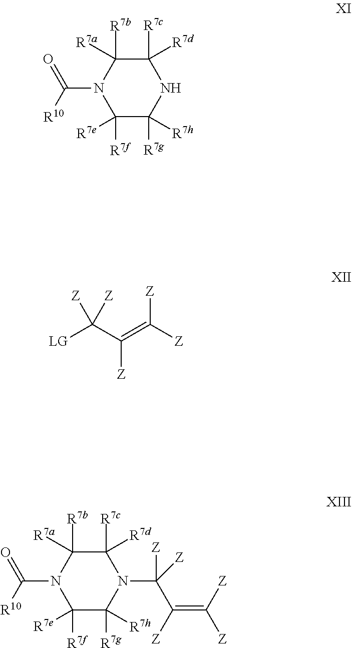

wherein R¹⁰ is a substituted or unsubstituted linear or branched alkyl group, or a substituted or unsubstituted linear or branched alkoxy group, and
LG is leaving group;
(b) reacting a compound having the structure XIII with a first oxidizing agent with heating to produce a compound having the structure XIV

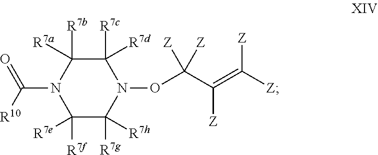

(c) reacting a compound having the structure XIV with (i) a second oxidizing agent followed by (ii) a first reducing agent to produce a first intermediate; and
(d) reacting the first intermediate with a second reducing agent to produce a compound having the structure X.

Aspect 48. The method of Aspect 47, wherein R¹⁰ is a C1-C5 linear or branched alkyl group.

Aspect 49. The method of Aspect 47, wherein R¹⁰ is a C1-C5 linear or branched alkoxy group.

Aspect 50. The method of Aspect 47, wherein R¹⁰ is a C1-C5 linear or branched alkoxy group substituted with an aryl group.

Aspect 51. The method of Aspect 47, wherein R¹⁰ is a benzyloxy group.

Aspect 52. The method of any one of Aspects 46-51, wherein LG is a halide, sulfonate, carbonate, or phosphate.

Aspect 53. The method of any one of Aspects 47-52, wherein the base in step (a) comprises a carbonate, hydroxide, phosphate, hydride, dialkylamide, or hexamethyldisilazide.

Aspect 54. The method of any one of Aspects 47-53, wherein step (a) is conducted in an aprotic organic solvent.

Aspect 55. The method of any one of Aspects 47-54, wherein step (a) is conducted at a temperature of from about 25° C. to about 100° C.

Aspect 56. The method of any one of Aspects 47-55, wherein step (b) comprises the steps of
(i) reacting a compound having the structure XIII with a first oxidizing agent in a first organic solvent to produce a first composition;
(ii) adding an aqueous base to the first composition to produce a second composition comprising an organic layer and an aqueous layer;
(iii) separating the organic layer from the aqueous layer;
(iv) removing the first organic solvent from the organic layer to produce a residue;
(v) dissolving the residue in a second organic solvent to produce a second composition; and
(vi) heating the second composition from about 50° C. to about 100° C. to produce the compound having the structure XIV.

Aspect 57. The method of Aspect 56, wherein the first organic solvent is dichloromethane and the second organic solvent is toluene

Aspect 58. The method of any one of Aspects 47-57, wherein the first oxidizing agent in step (b) comprises a peroxyacid, oxone, or hydrogen peroxide/acetic acid.

Aspect 59. The method of any one of Aspects 47-57, wherein the first oxidizing agent in step (b) comprises meta-chloroperoxybenzoic acid.

Aspect 60. The method of any one of Aspects 47-57, wherein the molar ratio of the first oxidizing agent to the compound having the structure XIII is from 0.95:1 to 1:1.05.

Aspect 61. The method of any one of Aspects 47-60, wherein step (c) comprises the steps of
(i) reacting a compound having the structure XIV with a second oxidizing agent in a third organic solvent to produce a third composition; and
(ii) mixing the first reducing agent with the third composition to produce the first intermediate.

Aspect 62. The method of Aspect 61, wherein the second oxidizing agent comprises ozone or osmium tetroxide with sodium metaperiodate.

Aspect 63. The method of Aspect 61 or 62, wherein the third organic solvent comprises an alcohol and an aprotic solvent.

Aspect 64. The method of any one of Aspects 61-63, wherein the compound having the structure XIV is reacted with the second oxidizing agent at a temperature of from about −50° C. to about −100° C.

Aspect 65. The method of any one of Aspects 47-64, wherein the first reducing agent comprises a hydride.

Aspect 66. The method of any one of Aspects 47-64, wherein the first reducing agent comprises a borohydride.

Aspect 67. The method of any one of Aspects 47-66, wherein the molar ratio of the first reducing agent to the compound having the structure XIV is from 1.5:1 to 2.5:1.

Aspect 68. The method of any one of Aspects 47-67, wherein the first intermediate is isolated prior to step (d).

Aspect 69. The method of any one of Aspects 47-68, wherein step (d) comprises the steps of
(i) dissolving the first intermediate in an aprotic solvent to produce a fourth composition;
(ii) mixing the second reducing agent with the fourth composition to produce the compound having the structure X; and
(iii) isolating and purifying the compound having the structure X.

Aspect 70. The method of Aspect 69, wherein the second reducing agent is mixed with the fourth composition at a temperature of from about 10° C. to about −50° C.

Aspect 71. The method of any one of Aspects 47-70, wherein the second reducing agent comprises a hydride.

Aspect 72. The method of any one of Aspects 47-70, wherein the second reducing agent comprises an aluminum hydride.

Aspect 73. The method of any one of Aspects 47-70, wherein the molar ratio of the second reducing agent to the first intermediate is from 4:1 to 2:1.

Aspect 74. The method of any one of Aspects 47-73, wherein R^{7a}, R^{7b}, R^{7c}, R^{7d}, R^{7e}, R^{7f}, R^{7g}, and R^{7h} are each hydrogen.

Aspect 75. The method of any one of Aspects 47-73, wherein R⁵ is methyl and R^{7a}, R^{7b}, R^{7c}, R^{7d}, R^{7e}, R^{7f}, R^{7g}, and R^{7h} are each hydrogen.

Aspect 76. A method for making a compound having the structure XX

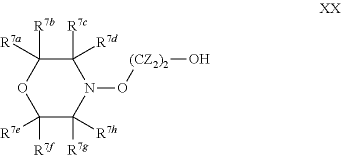

wherein
   R^{7a}, R^{7b}, R^{7c}, R^{7d}, R^{7e}, R^{7f}, R^{7g}, and R^{7h} are independently hydrogen, deuterium, or a substituted or unsubstituted alkyl group; and
   each Z is independently hydrogen or deuterium,
the method comprising
   (a) reacting a compound having the structure XXI with a compound having the structure XXII in the presence of a base to produce a compound having the structure XXIII

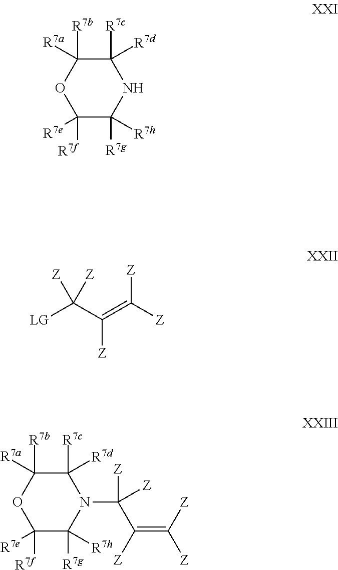

wherein LG is leaving group;
(b) reacting a compound having the structure XXIII with a first oxidizing agent to produce a compound having the structure XXIV

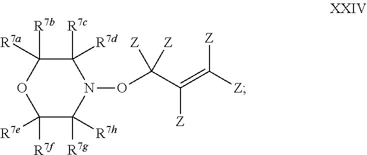

(c) reacting the compound having the structure XXIV with (i) a second oxidizing agent followed by (ii) a first reducing agent to produce a compound having the structure XX.

Aspect 77. The method of Aspect 76, wherein LG is a halide, sulfonate, carbonate, or phosphate.

Aspect 78. The method of Aspect 76 or 77, wherein the base in step (a) comprises a carbonate, hydroxide, phosphate, hydride, dialkylamide, or hexamethyldisilazide.

Aspect 79. The method of any one of Aspects 76-78, wherein step (a) is conducted in an aprotic organic solvent.

Aspect 80. The method of any one of Aspects 76-79, wherein step (a) is conducted at a temperature of from about 25° C. to about 100° C.

Aspect 81. The method of any one of Aspects 76-80, wherein step (b) comprises the steps of
(i) reacting a compound having the structure XXIII with a first oxidizing agent in a first organic solvent to produce a first composition;
(ii) adding an aqueous base to the first composition to produce a second composition comprising an organic layer and an aqueous layer;
(iii) separating the organic layer from the aqueous layer;
(iv) removing the first organic solvent from the organic layer to produce a residue;
(v) dissolving the residue in a second organic solvent to produce a second composition; and
(vi) heating the second composition from about 50° C. to about 100° C. to produce the compound having the structure XXIV.

Aspect 82. The method of Aspect 81, wherein the first organic solvent is dichloromethane and the second organic solvent is toluene

Aspect 83. The method of any one of Aspects 76-82, wherein the first oxidizing agent in step (b) comprises a peroxyacid, oxone, or hydrogen peroxide/acetic acid.

Aspect 84. The method of any one of Aspects 76-83, wherein the first oxidizing agent in step (b) comprises meta-chloroperoxybenzoic acid.

Aspect 85. The method of any one of Aspects 76-83, wherein the molar ratio of the first oxidizing agent to the compound having the structure XIII is from 0.95:1 to 1:1.05.

Aspect 86. The method of any one of Aspects 76-84, wherein step (c) comprises the steps of
(i) reacting a compound having the structure XXIV with a second oxidizing agent in a third organic solvent to produce a third composition; and
(ii) mixing the first reducing agent with the third composition to produce the compound having the structure XX; and
(iii) isolating and purifying the compound having the structure XX.

Aspect 87. The method of Aspect 86, wherein the second oxidizing agent comprises ozone or osmium tetroxide with sodium metaperiodate.

Aspect 88. The method of Aspect 86 or 87, wherein third organic solvent comprises an alcohol and an aprotic solvent.

Aspect 89. The method of any one of Aspects 86-88, wherein the compound having the structure XXIV is reacted with the second oxidizing agent at a temperature of from about −50° C. to about −100° C.

Aspect 90. The method of any one of Aspects 76-89, wherein the first reducing agent comprises a hydride.

Aspect 91. The method of any one of Aspects 76-89, wherein the first reducing agent comprises a borohydride.

Aspect 92. The method of any one of Aspects 76-91, wherein the molar ratio of the first reducing agent to the compound having the structure XXIV is from 1.5:1 to 2.5:1.

Aspect 93. The method of any one of Aspects 76-92, wherein R^{7a}, R^{7b}, R^{7c}, R^{7d}, R^{7e}, R^{7f}, R^{7g}, and R^{7h} are each hydrogen.

Aspect 94. The method of any one of Aspects 47-73, wherein R⁵ is methyl and R^{7a}, R^{7b}, R^{7c}, R^{7d}, R^{7e}, R^{7f}, R^{7g}, and R^{7h} are each hydrogen.

Aspect 95. A compound having the structure XXX

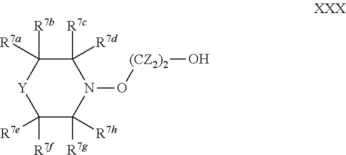

wherein
   Y is O or NR⁵,
   R⁵ is a substituted or unsubstituted linear or branched alkyl group, and
   R^{7a}, R^{7b}, R^{7c}, R^{7d}, R^{7e}, R^{7f}, R^{7g}, and R^{7h} are independently hydrogen, deuterium, or a substituted or unsubstituted alkyl group; and
each Z is independently hydrogen or deuterium.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure.

### Example 1

General Experimental All reactions were conducted in oven dried glassware capped with a rubber septum under an argon atmosphere unless otherwise stated. All organic solutions were concentrated under reduced pressure on a rotary evaporator and water bath. Flash-column chromatography was performed using silica gel (Fisher Silica Gel Sorbent (230-400 Mesh, Grade 60)). Thin-layer chromatography (TLC) was carried out with 250 μM glass back silica (XHL) plates with fluorescent indicator (254 nm). TLC plates were visualized by exposure to ultraviolet light (UV) and/or submersion in ceric ammonium molybdate (CAM) in ethanol followed by heating on a hot plate (120° C., 10-15 s).

Materials Starting material 2-(tert-butyldimethylsilyloxy)-ethan-1-ol was purchased from AKScientific. 2-Hydroperoxy-2-methyltetrahydro-2H-pyran (MTHPOOH) was prepared according to literature procedures¹¹⁻¹³ and spectral data were in accord with that previously reported in the literature. Morpholine and N-methylpiperazine were purchased from Sigma-Aldrich. Isopropylmagnesium chloride lithium chloride complex solution (1.3 M in THF) was purchased from Sigma-Aldrich. Ethylmagnesium bromide (3M in THF) was purchased from Sigma-Aldrich. Tetrabutylammonium fluoride (1 M in THF) was purchased from TCI. Thionyl chloride was purchased from Alfa Aesar. Sodium Hydride (60% dispersion in mineral oil) was purchased from Sigma-Aldrich. 4-(3-Chloro-4-fluorophenylamino)-7-methoxyquinazolin-6-ol was purchased from AK Scientific. 4-(3-Chloro-2-fluorophenylamino)-7-methoxyquinazolin-6-ol was purchased from AmBeed. Gefitinib (1) (≥98% (HPLC), SML1657-50MG) was purchased from Sigma-Aldrich. Materials for in vitro assays are given in the “In vitro assay methods” section of the Examples section. The purity of the assayed compounds (6, 13-15) were determined to be >95% by UHPLC and NMR.

Instrumentation Nuclear Magnetic resonance (NMR) spectra of all compounds were obtained in either CDCl₃ (δ 7.26 and 77.16 ppm, respectively) or DMSO-D₆ (δ 2.50 and 39.52 ppm, respectively) using a 500 MHz, EZC500 JEOL instrument at 25° C. The chemical shifts (6) are calculated with respect to residual solvent peak and are given in ppm. Multiplicities are abbreviated as follows: s (singlet), m (multiplet), b (broad), d (doublet), t (triplet), q (quartet), hept (heptet). High resolution mass spectra were obtained on a ThermoFisher Orbitrap Q-Exactive using electrospray ionization (ESI). Melting points were determined on a Barstead Electrothermal 9100. UHPLC traces of 8, 13-15 were obtained using a ThermoFisher Vanquish UHPLC with PDA detector and an Acclaim 120 ¹⁸C 4.6×50 mm column and the % purity determined using the Avalon peak area algorithm. Instrumentation used in the in vitro assays are given in the in vitro assay methods section of the Examples Section.

### Synthesis of tert-butyldimethyl(2-((2-methyltetrahydro-2H-pyran-2-yl)peroxy)ethoxy)silane (8) by literature procedure¹⁴

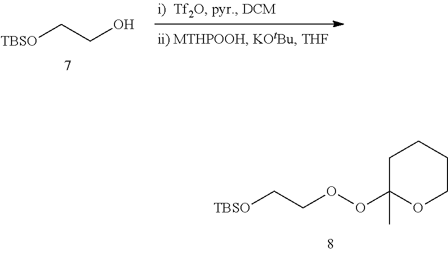 [SMILES: CC(C)(C)[Si](C)(C)OCCO ; CC1(OOCCO[Si](C)(C)C(C)(C)C)CCCCO1]

To a stirred solution of 7 (13.33 g, 75.71 mmol, 1.0 eq.) in anhydrous DCM (250 mL) at 0° C. was added pyridine (9.66 mL, 113.57 mmol, 1.5 eq.) followed by dropwise addition of Tf₂O (15.26 mL, 90.85 mmol, 1.2 eq.) and the solution was stirred for 45 min at 0° C. After such time, the mixture was diluted with DCM (100 mL) and washed successively with 1 N HCl (1×, 150 mL), aq. NaHCO₃ (1×, 150 mL) and aq. brine (1×, 150 mL). The combined organic layers were dried over Na₂SO₄, filtered and concentrated in-vacuo. The crude triflate was used directly in the next step without further purification.

To a stirred solution of MTHPOOH¹¹⁻¹³ (12 g, 90.85 mmol, 1.2 eq.) in anhydrous THF (260 mL) at 0° C. was added KO^{t}Bu (10.19 g, 90.85 mmol, 1.2 eq.) and the solution was stirred for 30 min, after which, the crude triflate obtained in the previous step was added dropwise in anhydrous THF (40 mL). The solution was warmed to r.t. and stirred for 90 min. After such time the mixture was diluted with EtOAc (100 mL) and quenched via addition of NaHCO₃ (1×, 200 mL). The layers were separated and the aqueous layer extracted with EtOAc (2×, 100 mL). Combined organic layers were dried over Na₂SO₄, filtered and concentrated in-vacuo. Residue obtained was purified by flash column chromatography on silica (eluent: 10:90 EtOAc:Hexanes) to afford the title compound 8 (11.19 g, 38.56 mmol, 51%) as an off yellow oil. Spectral data are in accord with that previously reported in the literature.¹⁴

R_{f}=0.50 (20:80 EtOAc:Hexanes; CAM).

¹H NMR (500 MHz, CDCl₃): δ 4.13-4.07 (m, 2H), 3.92 (td, J=11.4, 2.8 Hz, 1H), 3.85 (t, J=5.4 Hz, 2H), 3.71-3.68 (m, 1H), 1.79-1.50 (m, 6H), 1.43 (s, 3H), 0.90 (s, 9H), 0.08 (s, 6H).

¹³C NMR (126 MHz, CDCl₃): δ 102.5, 76.7, 61.8, 60.8, 33.3, 26.0, 24.9, 24.6, 19.2, 18.5, −5.2.

### Synthesis of 4-(2-((tert-butyldimethylsilyl)oxy)ethoxy)morpholine (10)

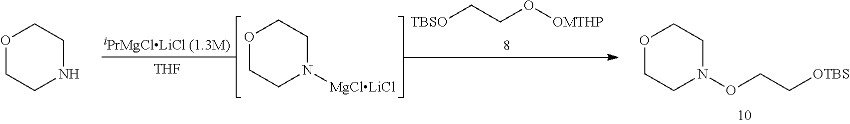

To a stirred solution of morpholine (7.64 mL, 87.33 mmol, 3.0 eq.) in anhydrous THF (73 mL) at 0° C. was added ⁱPrMgCl·LiCl (1.3M in THF) (56 mL, 72.78 mmol, 2.5 eq.) and the solution brought to r.t. and stirred for 45 min. After such time, 8 (8.45 g, 29.11 mmol, 1.0 eq.) was added dropwise in anhydrous THF (73 mL) and the solution was stirred for 3 h. After such time, the solution was quenched with NaHCO₃ (100 mL). The layers were separated and the aqueous layer extracted with EtOAc (3×, 75 mL). Combined organic layers were dried over Na₂SO₄, filtered and concentrated in-vacuo. The residue obtained was purified by flash column chromatography on silica (eluent: 15:85 EtOAc:Hexanes) to afford the title compound 10 (4.95 g, 18.95 mmol, 65%) as a light yellow oil.

R_{f}=0.20 (10:90 EtOAc:Hexanes; CAM).

¹H NMR (500 MHz, CDCl₃): δ 3.88 (d, J=11.8 Hz, 2H), 3.79-3.72 (m, 4H), 3.62-3.54 (m, 2H), 3.15 (d, J=10.6 Hz, 2H), 2.66 (td, J=10.9, 3.3 Hz, 2H), 0.89 (s, 9H), 0.06 (s, 6H).

¹³C NMR (126 MHz, CDCl₃): δ 73.1, 66.4, 61.6, 56.4, 26.0, 18.5, -5.1.

HRMS-ESI (m/z): [M+H]⁺ calculated for [C₁₂H₂₈O₃N²⁸Si]⁺: 262.1833. found: 262.1835.

### 1-(2-((tert-butyldimethylsilyl)oxy)ethoxy)-4-methylpiperazine (16)

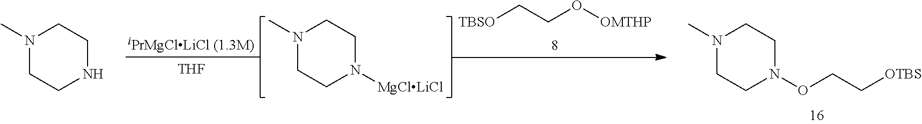

To a stirred solution of N-methylpiperazine (12.62 mL, 113.73 mmol, 3.0 eq.) in anhydrous THF (95 mL) at 0° C. was added ⁱPrMgCl·LiCl (1.3M in THF) (72.9 mL, 94.78 mmol, 2.5 eq.) and the solution brought to r.t. and stirred for 45 min. After such time, 8 (11 g, 37.91 mmol, 1.0 eq.) was added dropwise in anhydrous THF (95 mL) and the solution was stirred for 3 h. After such time, the solution was quenched with NaHCO₃ (75 mL). The layers were separated and the aqueous layer extracted with EtOAc (3×, 50 mL). The combined organic layers were dried over Na₂SO₄, filtered and concentrated in-vacuo. The residue obtained was purified by flash column chromatography on silica (eluent: 40:55:5 Hexanes:EtOAc:Et₃N) to afford the title compound 16 (6.99 g, 25.5 mmol, 67%) as a yellow oil.

R_{f}=0.30 (40:55:5 Hexanes:EtOAc:Et₃N; CAM).

¹H NMR (500 MHz, CD₆) δ 3.83 (t, J=5.2 Hz, 2H), 3.75 (t, J=5.5 Hz, 2H), 3.15-3.13 (m, 2H), 2.84 (brs, 2H), 2.49-2.46 (m, 2H), 2.10-2.06 (m, 2H), 2.02 (s, 3H), 0.99 (s, 9H), 0.09 (s, 6H).

¹³C NMR (126 MHz, C₆D₆) δ 73.2, 62.0, 55.9, 54.4, 45.6, 26.2, 18.6, -5.1.

HRMS-ESI (m/z): [M+H]⁺ calculated for [C₁₃H₃₁O₂N₂Si]⁺: 275.2149. found: 275.2146.

### Synthesis of 2-(morpholinooxy)ethan-1-ol (11)

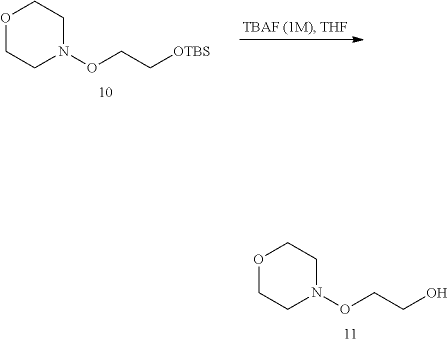 [SMILES: CC(C)(C)[Si](C)(C)OCCON1CCOCC1 ; OCCON1CCOCC1]

To a stirred solution of 10 (4.90 g, 18.80 mmol, 1.0 eq.) in anhydrous THF (150 mL) at 0° C. was added TBAF (1 M in THF) (37.60 mL, 37.60 mmol, 2.0 eq.) dropwise and the solution was brought to r.t. and stirred for 1 h. After such time, the mixture was concentrated in-vacuo and the residue obtained was purified by flash column chromatography (eluent: 95:5 EtOAc:Et₃N) to afford the title compound 11 (2.17 g, 14.75 mmol, 79%) as a yellow oil.

R_{f}=0.50 (95:5 EtOAc:Et₃N; CAM)

¹H NMR (500 MHz, CDCl₃): δ 3.91 (d, J=12.3 Hz, 2H), 3.86-3.79 (m, 4H), 3.56 (t, J=12.6 Hz, 2H), 3.34 (br s, 1H), 3.21 (d, J=11.9 Hz, 2H), 2.67 (td, J=10.9, 3.3 Hz, 2H).

¹³C NMR (126 MHz, CDCl₃): δ 71.6, 66.3, 63.6, 56.2.

HRMS-ESI (m/z): [M+H]⁺ calculated for [C₆H₁₄O₃N]⁺: 148.0968. found: 148.0964.

### 2-((4-methylpiperazin-1-yl)oxy)ethan-1-ol (17)

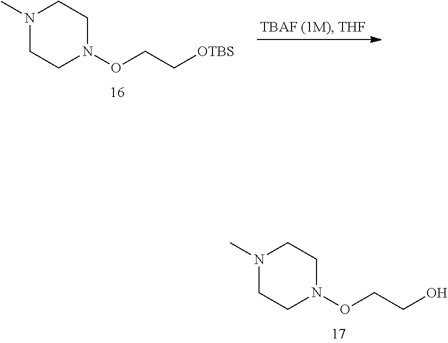 [SMILES: CN1CCN(OCCO[Si](C)(C)C(C)(C)C)CC1 ; CN1CCN(OCCO)CC1]

To a stirred solution of 16 (6.35 g, 23.16 mmol, 1.0 eq.) in anhydrous THF (300 mL) at 0° C. was added TBAF (1 M in THF) (46.32 mL, 46.32 mmol, 2.0 eq.) dropwise and the solution was brought to r.t. and stirred for 90 min. After such time, the mixture was concentrated in-vacuo and the residue obtained was purified by flash column chromatography (eluent: 90:10 EtOAc:Et₃N) to afford the title compound 17 (2.18 g, 13.61 mmol, 71%) as a yellow oil.

R_{f}=0.10 (90:10 EtOAc:Et₃N; CAM).

¹H NMR (500 MHz, Toluene-D₆) δ 3.69-3.66 (m, 2H), 3.65-3.60 (m, 2H), 3.01 (br d, J=10.3 Hz, 2H), 2.73-2.59 (m, 2H), 2.41 (br d, J=11.3 Hz, 2H), 1.96 (s, 3H), 1.94-1.90 (m, 2H).

¹³C NMR (126 MHz, Toluene-D₈) δ 72.2, 63.2, 55.4, 54.2, 45.3.

HRMS-ESI (m/z): [M+H]⁺ calculated for [C₇H₁₇O₂N₂]⁺: 161.1285. found: 161.1282.

### Synthesis of N-(3-chloro-4-fluorophenyl)-7-methoxy-6-(2-(morpholinooxy)ethoxy)quinazolin-4-amine (6)

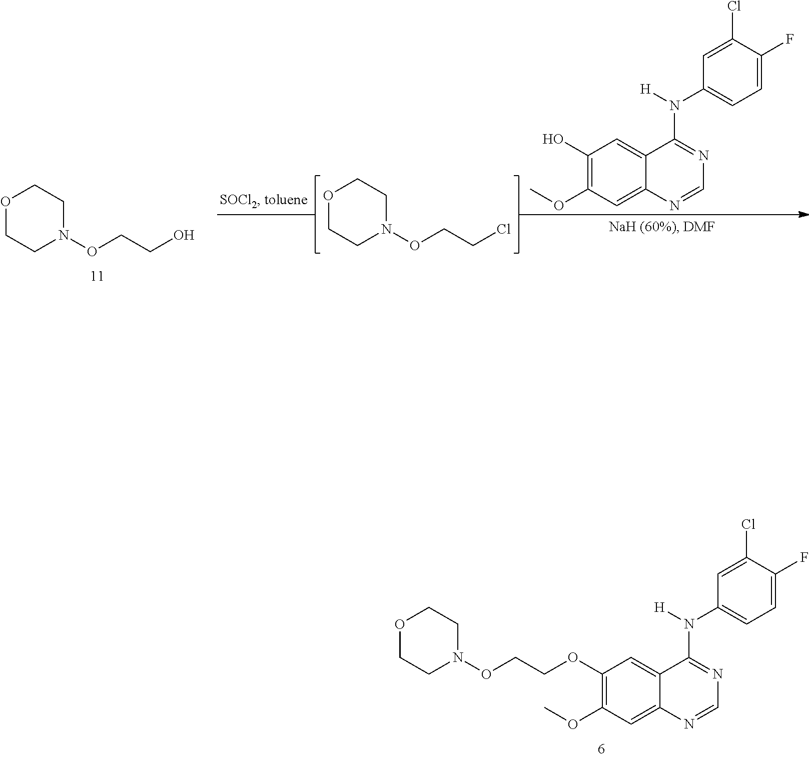

To a stirred solution of 11 (1 g, 6.80 mmol, 1.0 eq.) in anhydrous toluene (20 mL) at 0° C. was added SOCl₂ (1.23 mL, 17.00 mmol, 2.5 eq.) dropwise, stirred until no longer exothermic and then brought to 60° C. and stirred for 3 h. After such time, the mixture was concentrated in-vacuo and the resulting residue was used directly in the next step without further purification.

To a stirred solution of 4-(3-chloro-4-fluorophenylamino)-7-methoxyquinazolin-6-ol (3.26 g, 10.20 mmol, 1.5 eq.) in anhydrous DMF (35 mL) at 0° C. was added NaH (60% dispersion in mineral oil) (406 mg, 10.20 mmol, 1.5 eq.) slowly and the solution was warmed to r.t. and stirred for 45 min. Following this, the crude chloride obtained in the previous step was added dropwise in DMF (5 mL) and the solution brought to 80° C. and stirred for 16 h. After such time the mixture was concentrated in-vacuo and co-concentrated with toluene (3×, 50 mL). The resulting residue was then redissolved in EtOAc (200 mL) and washed successively with 1 M NaOH (2×, 100 mL), brine (2×, 100 mL), then the combined organic layers were dried over Na₂SO₄, filtered and concentrated in-vacuo. The residue obtained was purified by flash column chromatography (eluent: 10:90 Et₃N:EtOAc) to afford the title compound 6 (1.08 g, 2.41 mmol, 35% over 2 steps) as a light yellow powder.

R_{f}=0.50 (10:90 Et₃N:EtOAc; UV, CAM)

¹H NMR (500 MHz, DMSO-D₆): δ 9.51 (s, 1H), 8.50 (s, 1H), 8.12 (dd, J=6.8, 2.7 Hz, 1H), 7.83-7.78 (m, 2H), 7.44 (t, J=9.1 Hz, 1H), 7.20 (s, 1H), 4.29 (t, J=4.5 Hz, 2H), 4.08 (t, J=4.5 Hz, 2H), 3.94 (s, 3H), 3.81 (d, J=11.6 Hz, 2H), 3.44 (t, J=11.4 Hz, 2H) 3.18 (d, J=10.4 Hz, 2H), 2.54 (d, J=11.8 Hz, 2H).

¹³C NMR (126 MHz, DMSO-D₆): δ 156.0, 154.5, 153.1 (d, ¹J_{C-F}=243.2 Hz), 152.7, 148.2, 147.0, 136.8 (d, ³J_{C-F}=3.8 Hz), 123.4, 122.2 (d, ³J_{C-F}=6.3 Hz), 118.8 (d, ²J_{C-F}=18.9f Hz), 116.5 (d, ²J_{C-F}=21.4 Hz), 108.7, 107.4, 102.7, 69.0, 67.4, 65.5, 56.2, 55.8.

¹³C NMR {¹⁹F} (126 MHz, DMSO-D₆): δ 156.0, 154.5, 153.1, 152.7, 148.2, 147.0, 136.8, 123.4, 122.2, 118.8, 116.5, 108.7, 107.4, 102.7, 69.0, 67.4, 65.5, 56.2, 55.8.

¹⁹F {¹H} (470 MHz, DMSO-D₆): −123.2

HRMS-ESI (m/z): [M+H]⁺ calculated for [C₂₁H₂₃O₄N₄³⁵ClF]⁺: 449.1386. found: 449.1378.

m.p: 186.4-187.5° C. (mean of n=3 determinations)

### Synthesis of N-(3-chloro-2-fluorophenyl)-7-methoxy-6-(2-(morpholinooxy)ethoxy)quinazolin-4-amine (13)

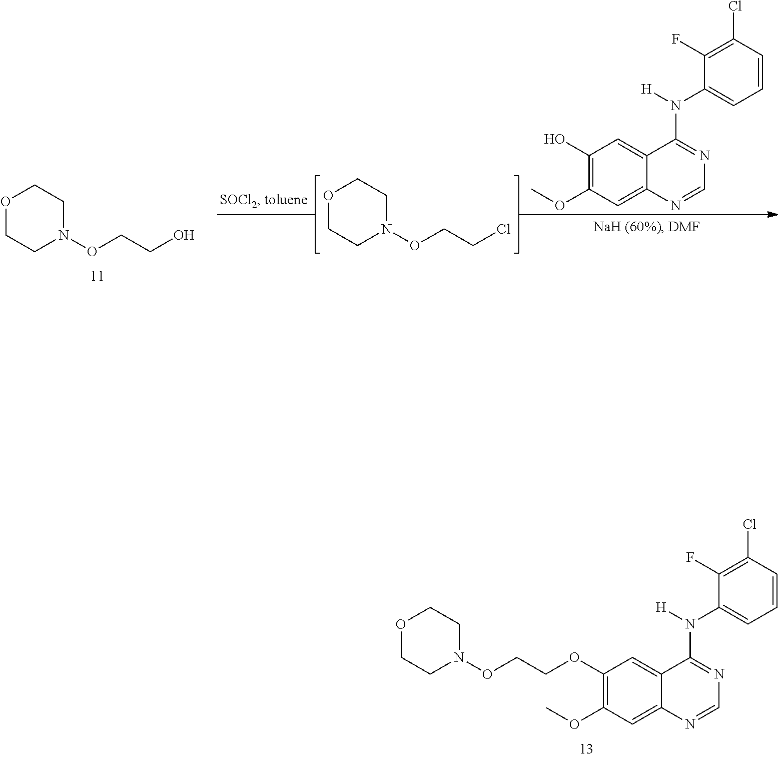

To a stirred solution of 11 (1.90 g, 12.9 mmol, 1.0 eq.) in anhydrous toluene (40 mL) at 0° C. was added SOCl₂ (3.84 mL, 32.25 mmol, 2.5 eq.) dropwise, stirred until no longer exothermic and then brought to 60° C. and stirred for 3 h. After such time, the mixture was concentrated in-vacuo and the resulting residue was used directly in the next step without further purification.

To a stirred solution of 4-(3-chloro-2-fluorophenylamino)-7-methoxyquinazolin-6-ol (6.19 g, 19.35 mmol, 1.5 eq.) in anhydrous DMF (70 mL) at 0° C. was added NaH (60% dispersion in mineral oil) (770 mg, 19.35 mmol, 1.5 eq.) slowly and the solution was warmed to r.t. and stirred for 45 min. Following this, the crude chloride obtained in the previous step was added dropwise in DMF (10 mL) and the solution brought to 80° C. and stirred for 16 h. After such time the mixture was concentrated in-vacuo and co-concentrated with toluene (3×, 75 mL). The resulting residue was then redissolved in EtOAc (150 mL) and washed successively with 1 M NaOH (2×, 75 mL), brine (2×, 75 mL), then the combined organic layers were dried over Na₂SO₄, filtered and concentrated in-vacuo. The residue obtained was purified by flash column chromatography (eluent: 10:90 Et₃N:EtOAc) to afford the title compound 13 (1.67 g, 3.73 mmol, 29% over 2 steps) as an off white powder.

R_{f}=0.50 (10:90 Et₃N:EtOAc; UV, CAM)

¹H NMR (500 MHz, DMSO-D₆): δ 9.61 (s, 1H), 8.38 (s, 1H), 7.82 (s, 1H), 7.54-7.52 (m, 1H), 7.49-7.46 (m, 1H), 7.28 (t, J=8.3 Hz, 1H), 7.21 (s, 1H), 4.28 (t, J=4.6 Hz, 2H), 4.07 (t, J=4.6 Hz, 2H), 3.94 (s, 3H), 3.81 (d, J=12.0 Hz, 2H), 3.44 (t, J=11.3 Hz, 2H), 3.17 (d, J=10.4 Hz, 2H), 2.55-2.52 (m, 2H).

¹³C NMR (126 MHz, DMSO-D₆): δ 156.9, 154.6, 153.0, 152.4 (d, ¹J_{C-F}=249.5 Hz), 148.2, 147.0, 128.4 (d, ²J_{C-F}=12.6 Hz), 127.1, 126.9, 124.9 (d, ³J_{C-F}=5.0 Hz), 120.2 (d, ²J_{C-F}=16.4 Hz), 108.6, 107.2, 102.8, 69.0, 67.3, 65.5, 56.2, 55.9.

¹³C NMR {¹⁹F} (126 MHz, DMSO-D₆): δ 156.9, 154.6, 153.0, 152.4, 148.2, 147.0, 128.4, 127.1, 126.9, 124.9, 120.2, 108.6, 107.2, 102.8, 69.0, 67.3, 65.5, 56.2, 55.9.

¹⁹F {¹H} (470 MHz, DMSO-D₆): −120.4.

HRMS-ESI (m/z): [M+H]⁺ calculated for [C₂₁H₂₃O₄N₄³⁵ClF]⁺: 449.1386. found: 449.1376.

### Synthesis of N-(3-chloro-4-fluorophenyl)-7-methoxy-6-(2-((4-methylpiperazin-1-yl)oxy)ethoxy)quinazolin-4-amine (14)

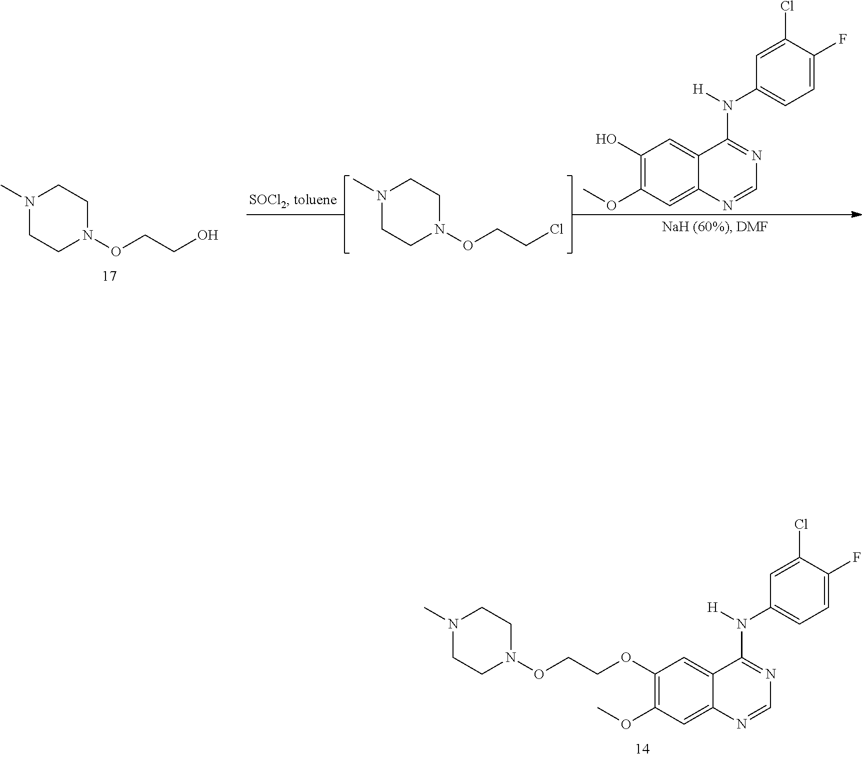

To a stirred solution of 17 (300 mg, 1.87 mmol, 1.0 eq.) in anhydrous toluene (7 mL) at 0° C. was added SOCl₂ (340 μL, 4.68 mmol, 2.5 eq.) dropwise, stirred until no longer exothermic and then brought to 60° C. and stirred for 3 h. After such time, the mixture was concentrated in-vacuo and the resulting residue was dissolved in EtOAc (20 mL) and washed with an aq. K₂CO₃ soln. (2×, 15 mL). The combined organic layers were then dried over Na₂SO₄, filtered and concentrated. The residue obtained was used without further purification.

To a stirred solution of 4-(3-chloro-4-fluorophenylamino)-7-methoxyquinazolin-6-ol (898 mg, 2.81 mmol, 1.5 eq.) in anhydrous DMF (10 mL) at 0° C. was added NaH (60% dispersion in mineral oil) (112 mg, 2.81 mmol, 1.5 eq.) slowly and the solution was warmed to r.t. and stirred for 45 min. Following this, the crude chloride obtained in the previous step was added dropwise in DMF (5 mL) and the solution brought to 80° C. and stirred for 16 h. After such time the mixture was concentrated in-vacuo and co-concentrated with toluene (3×, 20 mL). The resulting residue was then redissolved in EtOAc (30 mL) and washed successively with 1 M NaOH (2×, 20 mL), brine (2×, 20 mL), then the combined organic layers were dried over Na₂SO₄, filtered and concentrated in-vacuo. The residue obtained was purified by flash column chromatography (eluent: 85:10:5 EtOAc:MeOH:Et₃N) to afford the title compound 14 (286 mg, 0.687 mmol, 33% over 2 steps) as a light orange powder.

R_{f}=0.40 (85:10:5 EtOAc:MeOH:Et₃N; UV, CAM).

¹H NMR (500 MHz, DMSO-D₆): δ 9.53 (s, 1H), 8.50 (s, 1H), 8.12 (dd, J=6.8, 2.6 Hz, 1H), 7.84-7.78 (m, 2H), 7.44 (t, J=9.1 Hz, 1H), 7.21 (s, 1H), 4.28 (t, J=5.0 Hz, 2H), 4.04 (t, J=5.0 Hz, 2H), 3.94 (s, 3H), 3.15-3.13 (m, 2H), 2.70-2.67 (m, 2H), 2.57 (br s, 2H), 2.13-2.09 (m, 5H).

¹³C NMR (126 MHz, DMSO-D₆): δ 156.0, 154.5, 153.1 (d, ¹J_{C-F}=243.2 Hz), 152.7, 148.2, 147.0, 136.8, 126, 123.4, 122.2 (d, ³J_{C-F}=6.3 Hz), 118.8 (d, ²J_{C-F}=18.9 Hz), 116.5 (d, ²J_{C-F}=21.4 Hz), 108.7, 107.4, 102.7, 69.1, 67.4, 55.9, 55.0, 53.5, 45.0.

¹³C NMR {¹⁹F} (126 MHz, DMSO-D₆): δ 156.0, 154.5, 153.1, 152.7, 148.2, 147.0, 136.8, 123.4, 122.2, 118.8, 116.5, 108.7, 107.4, 102.7, 69.0, 67.4, 55.9, 55.0, 53.5, 45.0.

¹⁹F {¹H} (470 MHz, DMSO-D₆): −123.2.

HRMS-ESI (m/z): [M+H]⁺ calculated for [C₂₂H₂₆O₃N₅³⁵ClF]⁺: 462.1703. found: 462.1691.

m.p: 64.0-66.5° C. (mean of n=3 determinations)

### Synthesis of N-(3-chloro-2-fluorophenyl)-7-methoxy-6-(2-((4-methylpiperazin-1-yl)oxy)ethoxy)quinazolin-4-amine (15)

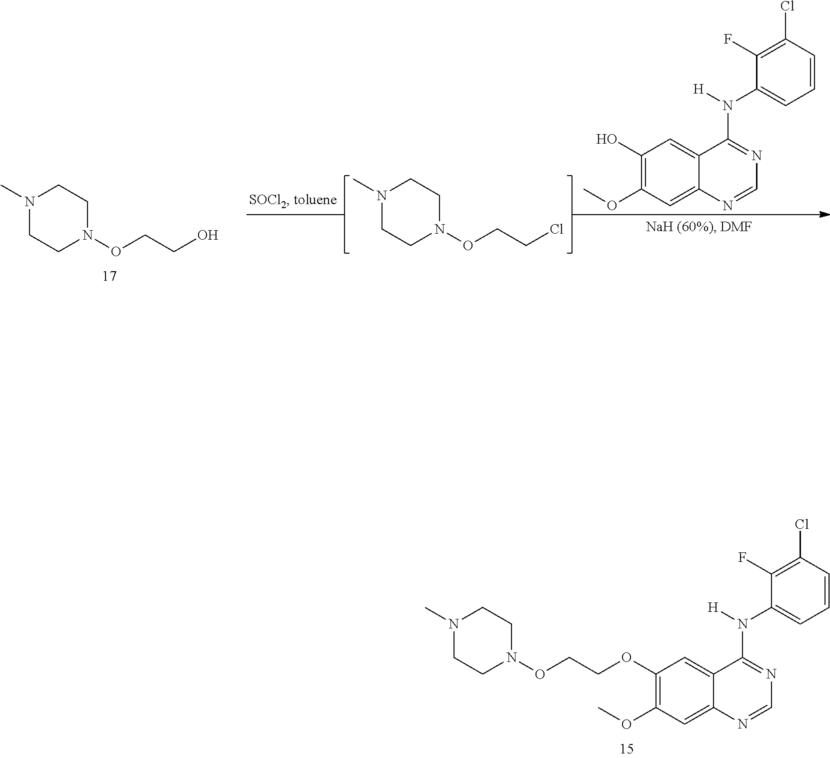

To a stirred solution of 17 (2.10 g, 13.12 mmol, 1.0 eq.) in anhydrous toluene (50 mL) at 0° C. was added SOCl₂ (2.38, mL, 32.80 mmol, 2.5 eq.) dropwise, stirred until no longer exothermic and then brought to 60° C. and stirred for 3 h. After such time, the mixture was concentrated in-vacuo and the resulting residue was dissolved in EtOAc (100 mL) and washed with an aq. K₂CO₃ soln. (2×, 75 mL). The combined organic layers were then dried over Na₂SO₄, filtered and concentrated. The residue obtained was used without further purification.

To a stirred solution of 4-(3-chloro-2-fluorophenylamino)-7-methoxyquinazolin-6-ol (6.29 g, 19.68 mmol, 1.5 eq.) in anhydrous DMF (90 mL) at 0° C. was added NaH (60% dispersion in mineral oil) (783 mg, 19.68 mmol, 1.5 eq.) slowly and the solution was warmed to r.t. and stirred for 45 min. Following this, the crude chloride obtained in the previous step was added dropwise in DMF (10 mL) and the solution brought to 80° C. and stirred for 16 h. After such time the mixture was concentrated in-vacuo and co-concentrated with toluene (3×, 25 mL). The resulting residue was then redissolved in EtOAc (150 mL) and washed successively with 1M NaOH (2×, 100 mL), aq. brine (2×, 100 mL), then the combined organic layers were dried over Na₂SO₄, filtered and concentrated in-vacuo. The residue obtained was purified by flash column chromatography (eluent: 85:10:5 EtOAc:Et₃N:MeOH) to afford the title compound 15 (1.19 g, 2.58 mmol, 20% over 2 steps) as a light yellow powder.

R_{f}=0.30 (85:10:5 EtOAc:MeOH:Et₃N; UV, CAM).

¹H NMR (500 MHz, DMSO-D₆): δ 9.61 (s, 1H), 8.39 (s, 1H), 7.83 (s, 1H), 7.57-7.51 (m, 1H), 7.48-7.46 (m, 1H), 7.29-7.26 (m, 1H), 7.21 (s, 1H), 4.27 (t, J=4.6 Hz, 2H), 4.03 (t, J=4.6 Hz, 2H), 3.94 (s, 3H), 3.15-3.12 (m, 2H), 2.69-2.67 (m, 2H), 2.57 (br s, 2H), 2.13-2.06 (m, 5H).

¹³C NMR (126 MHz, DMSO-D₆): δ 156.9, 154.6, 153.0, 152.4 (d, ¹J_{C-F}=249.5 Hz), 148.2, 147.0, 128.4 (d, ²J_{C-F}=11.3 Hz), 127.1, 126.8, 124.9 (d, ³J_{C-F}=5.0 Hz), 120.2 (d, ²J_{C-F}=16.4 Hz), 108.6, 107.2, 102.8, 69.0, 67.2, 55.9, 55.0, 53.6, 45.0.

¹³C NMR {¹⁹F} (126 MHz, DMSO-D₆): δ 156.9, 154.6, 153.0, 152.4, 148.2, 147.0, 128.4, 127.1, 126.8, 124.9, 120.1, 108.6, 107.2, 102.8, 69.0, 67.2, 55.9, 55.0, 53.6, 45.0.

¹⁹F {¹H} (470 MHz, DMSO-D₆): −120.4.

HRMS-ESI (m/z): [M+H]⁺ calculated for [C₂₂H₂₆O₃N₅³⁵ClF]⁺: 462.1703. found: 462.1695.

m.p: 143.5-144.9° C. (mean of n=3 determinations)

### Alternative Synthesis of Piperazinyl Hydroxylamines

An exemplary synthetic scheme for making piperazinyl analogue described herein is provided in FIG. 3. A [2,3]-Meisenheimer rearrangement is used to install the hydroxylamine unit.¹⁵⁻¹⁷ Beginning with commercially available 18 that on allylation with allyl bromide under basic conditions gave the N-allyl derivative (19) in 72% yield. N-oxidation of 19 with meta-chloroperoxybenzoic acid (MCPBA) followed by [2,3]-Meisenheimer rearrangement gave 20 in 47% yield over 2 steps. The alkene present in 20 was then subjected to ozonolysis followed by reduction with sodium borohydride (NaBH₄), after which, further reduction of the carboxybenzyl group with lithium aluminum hydride¹⁸ (LiAlH₄) gave the intended hydroxylamine precursor 17 in 50% yield over 2 steps.

### EXPERIMENTAL

### Synthesis of benzyl 4-allylpiperazine-1-carboxylate (19)

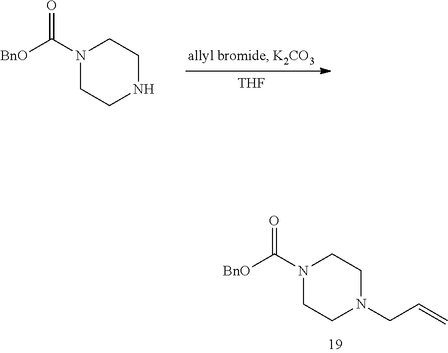 [SMILES: O=C(OCc1ccccc1)N1CCNCC1 ; C=CCN1CCN(C(=O)OCc2ccccc2)CC1 ; "THF"]

To a stirred solution of benzyl piperazine-1-carboxylate (15 g, 68.14 mmol, 1.0 eq.) in anhydrous THF (250 mL) at r.t. was added K₂CO₃ (18.8 g, 136.28 mmol, 2.0 eq.) followed by slow addition of allyl bromide (11.8 mL, 136.28 mmol, 2.0 eq.). The solution was brought to 65° C. and stirred for 16 h, after which time, the mixture was cooled to r.t. and quenched via addition of 1M NaOH (200 mL). The layers were separated and the aqueous layer extracted with EtOAc (3×, 100 mL). The combined organic layers were dried over Na₂SO₄, filtered and concentrated in vacuo. The residue obtained was purified by flash column chromatography on silica (eluent: 50:45:5 Hexanes:EtOAc:Et₃N) to afford the title compound 19 (12.81 g, 49.2 mmol, 72%) as a yellow oil. TLC R_{f}=0.60 (50:45:5 Hexanes:EtOAc:Et₃N; CAM, UV). ¹H NMR (500 MHz, CDCl₃) δ 7.37-7.29 (m, 5H), 5.89-5.78 (m, 1H), 5.23-5.14 (m, 2H), 5.13 (br s, 2H), 3.56 (m, 4H), 3.00 (d, J=6.6 Hz, 2H), 2.40 (br s, 4H). ¹³C NMR (126 MHz, CDCl₃) δ 155.4, 136.9, 134.7, 128.6, 128.1, 128.0, 118.5, 67.2, 61.8, 52.8, 43.9. HRMS-ESI (m/z) [M+H]⁺ calculated for [C₁₅H₂₁N₂O₂]⁺: 261.1598. found: 261.1594.

### Synthesis of benzyl 4-(allyloxy)piperazine-1-carboxylate (20)

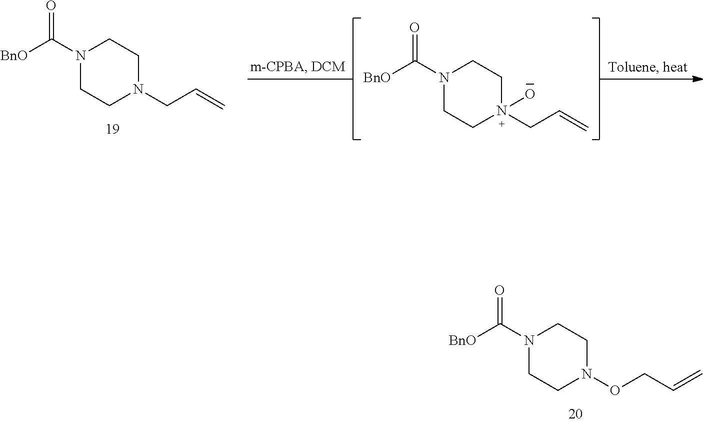

To a stirred solution of 19 (12.2 g, 46.9 mmol, 1.0 eq.) in anhydrous DCM at −30° C. was added MCPBA (70% mixture with 3-chlorobenzoic acid and water) (11.56 g, 46.9 mmol, 1.0 eq) portion-wise over a period of 5 min. After full addition, the reaction mixture was stirred at −30° C. for 1 h, then was quenched with aq. NaHCO₃ (150 mL) and the layers were separated. The organic layer was washed with NaHCO₃ (3×, 150 mL) after which the organic layer was dried over Na₂SO₄, filtered and concentrated in vacuo. The residue obtained was dissolved in toluene heated to 80° C. with stirring for 14 h. After such time, the mixture was cooled to r.t. and concentrated in vacuo. The residue obtained was purified by flash column chromatography on silica (eluent: 80:15:5 Hexanes:EtOAc:Et₃N) to afford the title compound 20 (6.13 g, 22.2 mmol, 47%) as a clear oil. TLC R_{f}=0.50 (30:70 EtOAc:Hexanes; CAM, UV). ¹H NMR (500 MHz, CDCl₃) δ 7.39-7.30 (m, 5H) 5.99-5.88 (m, 1H), 5.27 (d, J=17.3 Hz, 1H), 5.18 (d, J=10.3 Hz, 1H), 5.13 (s, 2H), 4.21 (d, J=6.1 Hz, 2H), 4.03 (s, 2H), 3.19-3.11 (m, 4H), 2.57 (s, 3H). ¹³C NMR (126 MHz, CDCl₃): δ 155.3, 136.8, 134.6, 128.7, 128.2, 128.1, 118.0, 73.2, 67.4, 55.3, 42.7. HRMS-ESI (m/z): [M+H]⁺ calculated for [C₁₅H₂₁N₂O₃]⁺: 277.1547. found: 277.1540.

### Synthesis of 2-((4-methylpiperazin-1-yl)oxy)ethan-1-ol (17)

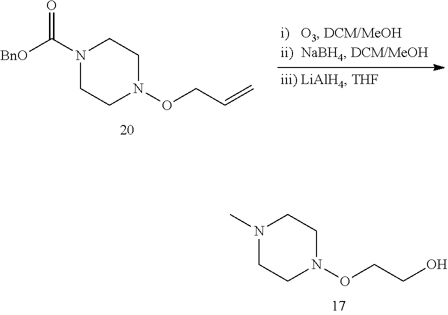 [SMILES: C=CCON1CCN(C(=O)OCc2ccccc2)CC1 ; CN1CCN(OCCO)CC1]

Compound 20 (5.6 g, 20.27 mmol, 1.0 eq.) was dissolved in a mixture of DCM (200 mL) and methanol (50 mL) and cooled to −78° C. Ozone was bubbled through the reaction mixture for 30 min until full consumption of starting material. On completion, as indicated by ESIMS, argon was bubbled through the reaction mixture to disperse residual ozone. NaBH₄ (1.53 g, 40.54 mmol, 2.0 eq.) was then added portion-wise at −78° C. and the solution was gradually warmed to r.t. and stirred for 1 h. After such time, the mixture was quenched with aq. NaHCO₃ (200 mL) and the layers separated. The aqueous layer was extracted with DCM (2×, 150 mL) and the combined organic layers washed with aq. brine (2×, 150 mL), dried over Na₂SO₄, filtered and concentrated in vacuo to directly afford the alcohol as a clear oil (5.26 g, 18.8 mmol, 93% crude yield). TLC R_{f}=0.10 (70:30 EtOAc:Hexanes; CAM, UV). ¹H NMR (500 MHz, CDCl₃) δ 7.38-7.30 (m, 5H), 5.13 (s, 2H), 4.07 (s, 2H), 3.84-3.81 (m, 4H), 3.24-3.10 (m, 5H), 2.60-2.56 (m, 2H). ¹³C NMR (126 MHz, CDCl₃) δ 155.2, 136.6, 128.7, 128.3, 128.1, 71.9, 67.5, 65.3, 55.0, 42.7. HRMS-ESI (m/z) [M+H]⁺ calculated for [C₁₄H₂₁O₄N₂]⁺: 281.1496. found: 281.1489.

The so obtained crude alcohol (5.26 g, 18.8 mmol, 1.0 eq.) obtained in the previous step was dissolved in anhydrous THF (68 mL) and cooled to 0° C. LiAlH₄ (2.31 g, 60.81 mmol, 3.2 eq.) was then added portion-wise over a period of 5 min. After full addition, the solution was warmed to r.t. and stirred for 2 h. After such time, the reaction mixture was cooled back to 0° C. and quenched by slow addition of H₂O. After effervescence ceased, the solution was acidified with concentrated HCl until pH≈2. The aqueous layer was then extracted with EtOAc (4×, 150 mL), after which, the aqueous layer was cooled to 0° C. and basified to pH≈10 by portion-wise addition of solid NaOH. The basified aqueous layer was extracted with EtOAc (4×, 100 mL) and the resulting organic layer was dried over Na₂SO₄, filtered and concentrated in vacuo to afford the title compound 17 (1.63 g, 10.2 mmol, 50% yield over 2 steps from 20) as a yellow oil with spectral data identical to that of an authentic sample.

### In Vitro Assay Methods

### Kinase Activity

In vitro kinase activity (inhibitor binding constants (K_{d}) and biochemical inhibition (IC₅₀'s)) were assessed by Eurofins DiscoverX (K_{d} determinations) and Eurofins Cerep (biochemical IC₅₀ determinations). For K_{d} determinations, compounds were ran in duplicate (n=2) and assayed using an 11-point, 3-fold dilution series at a top compound testing concentration of 10 μM with Eurofin's KINOMEScan KdELECT assay. For biochemical IC₅₀ determinations, compounds were ran in duplicate (n=2) and tested in an enzymatic radiometric assay using a 9-point, half-log dilution series at a top compound testing concentration of 10 μM and an ATP concentration of 10 μM with Eurofin's KinaseProfiler technology.

### Log D_{7.4} by Shake Flask Method (Pharmaron Inc., Ningbo, CN)

Compound lipophilicity was determined by Pharmaron Inc. using the shake-flask method with 1-octanol and PBS (pH 7.4). Each compound was assessed in duplicate (n=2) at 1 μM in a 96-well plate shaken at 25° C., 2,000 rpm for 2 h. Samples were analyzed by LC-MS/MS.

### Solubility Determinations in PBS at pH 7.4 (Pharmaron Inc., Ningbo, CN)

Solubility was determined by Pharmaron Inc. and each compound was assessed in duplicate (n=2) by adding 15 μL of compound stock solution (10 mM in DMSO) into a 96-well plate along with 485 μL of buffer followed by shaking at 25° C., 1,100 rpm for 2 h. Wells were filtered and samples (5 μL) were taken followed by dilution with an equal volume of DMSO (5 μL) and 490 μL of aqueous solution. Samples were analyzed by LC-MS/MS.

### Metabolic Stability Assay (Pharmaron Inc., Ningbo, CN)

Assessment of the compounds metabolic stability in incubations containing liver microsomes and hepatocytes of human and rat using the compound depletion approach was performed by Pharmaron Inc. Compound 15 underwent additional stability evaluation in monkey and dog hepatocytes. Microsome stability was assessed in duplicate (n=2) in the presence and absence of NADPH with liver microsomes (0.5 mg/mL) from human (BD Gentest) and SD rat (BD Gentest). Compounds were incubated in liver microsomes at 1 μM and samples (30 μL) were taken at 0.5, 15, 30, 45 and 60 min time points for analysis by LC-MS/MS. Hepatocyte stability was assessed in duplicate (n=2) at a working cell density of 0.5×10⁶ cells/mL with hepatocytes from human (BioIVT), SD rat (BioIVT), cynomolgous monkey (RILD) or beagle dog (BioIVT). Compounds were incubated in hepatocytes at 1 μM and samples (25 μL) were taken at 0.5, 15, 30, 60, 90 and 120 min time points for analysis by LC-MS/MS. The in vitro half-life and intrinsic clearances were determined as previously described.¹⁹

In vitro half-life (in vitro t_{1/2}) was determined from the slope value in: in vitro t_{1/2}=−(0.693/k).

Where, k, was determined by linear regression of the natural logarithm of the remaining percentage of the parent drug vs incubation time curve.

### Plasma Protein Binding by Equilibrium Dialysis (Pharmaron Inc., Ningbo, CN)

Plasma protein binding and brain tissue binding were determined by Pharmaron Inc. using the equilibrium dialysis method. Each compound was assessed in duplicate (n=2) at 5 μM with the final percent volume of organic solvent at 0.5%. Compounds were incubated at 37° C., 5% CO₂ for 6 h at 100 rpm and samples (50 μL) were taken at the beginning and end of the incubation. Following incubation, samples were analyzed by LC-MS/MS and the concentrations of compound were determined in the buffer and plasma solution chambers.

### Plasma Stability (Pharmaron Inc., Ningbo, CN)

Assessment of compounds to determine stability in incubations containing plasma of human (Pharmaron) and SD rat (IPHASE) were performed by Pharmaron Inc. using the compound deletion approach. Compounds were assessed in duplicate (n=2) and incubated in plasma at 5 μM and samples (50 μL) were taken at 0, 15, 30, 60 and 120 min time points for analysis by LC-MS/MS. Peak area ratios were determined from the extracted ion chromatogram and the percent compound remaining at each time point was calculated by the following equation.

Remaining percentage_{t min} (%)=peak area ratio_{t min}/peak area ratio_{0 min}×100

Where, peak area ratio_{t min} is peak area ratio of control and test compound at t min and peak area ratio_{0 min} is peak area ratio of control and test compound at zero time point.

### Permeability Studies (Pharmaron Inc., Ningbo, CN)

Compounds were evaluated at Pharmaron Inc. for their ability to permeate Caco-2 cells and MDCKII-MDR1 cells. A 96-well HTS Transwell plate (Corning) was used for the Caco-2 and MDCKII-MDR1 cell seeding. Caco-2 cells were seeded at a density of 6.86×10⁵ cells/mL and cultivated for 14-18 days prior to assays. MDCKII-MDR1 cells were seeded at a density of 1.56×10⁶ cells/mL and cultivated for 4-8 days prior to assays. To determine the rate of drug transport in both the absorptive (apical to basolateral (A-B)) and secretory (basolateral to apical (B-A)) directions, compounds (5 μM in DMSO for Caco-2 cells, and 1 μM in DMSO for MDCKII-MDR1 cells) were added to the donor wells. Plates were incubated at 37° C. for 2 h and samples (50 μL) were taken at the beginning and end of the incubation in both the donor and acceptor wells with the assay run in duplicate (n=2). Samples were analyzed by LC-MS/MS. The apparent permeability coefficient (Pₐₚₚ) in units (cm/s×10⁻⁶) was calculated using the following equation.

$${P}_{a p p} = {V}_{A} \times {[ drug ]}_{a c c e p t o r} / Area \times Time \times {[ drug ]}_{initial , donor}$$

where, V_{A} represents the volume (mL) in the acceptor well. Area is the surface of the membrane (0.143 cm² for Transwell-96 well plate) and time is the total transport time in sec.

The efflux ratio was determined using the following equation: Efflux ratio=Pₐₚₚ (B-A)/Pₐₚₚ(A-B)

Where, Pₐₚₚ (B-A) indicates the apparent permeability in the basolateral to apical direction, and Pₐₚₚ (A-B) indicates the apparent permeability in the apical to basolateral direction.

hERG Channel Inhibition Assay

The potential inhibitory effect of compounds on the hERG channel was assessed by Pharmaron Inc. using the manual patch clamp system as previously described.²⁰ The HEK293 cell line (Invitrogen) stably transfected with the hERG gene was employed. Compounds were tested at 5 concentrations (0.37, 1.11, 3.33, 10 and 30 μM) and run in triplicate (n=3). IC₅₀ values were determined by plotting the % inhibition against the concentration of compounds using GraphPad Prism from the non-linear regression equation fitted with a sigmoidal dose-response curve, and the IC₅₀ values are presented as the mean±SEM.

### Determination of Human CYP450 Inhibition by 15

Assessment of compound 15 was carried out at Pharmaron Inc. for its potential to inhibit cytochrome P450 (CYP) isoforms using human liver microsomes. The activities tested were CYP1A2-mediated phenacetin O-demethylation, CYP2C19-mediated (S)-mephenytoin 4′-hydroxylation, CYP2C9-mediated diclofenac 4′-hydroxylation, CYP2D6-mediated dextromethorphan O-demethylation and CYP3A4-mediated midazolam 1′-hydroxylation. Concentrations of substrates were phenacetin (40 μM), mephenytoin (50 μM), diclofenac (6 μM), dextromethorphan (2 μM) and midazolam (1 μM). Probe substrates phenacetin, mephenytoin and dextromethorphan were incubated at 37° for 20 min. Probe substrates diclofenac and midazolam were incubated at 37° for 5 min. Compound (15) was tested in an eight-point, threefold dilution series (0.01 μM to 30 μM) in DMSO and incubated with pooled human liver microsomes (0.5 mg/mL) (BD Gentest) and a cocktail of the probe substrates for selective CYP isoform. The reactions were initiated by adding NADPH (1 mM final concentration) after a 5 min-preincubation and the assay was ran in duplicate (n=2). Samples were analyzed by UPLC-MS/MS. Inhibition of each P450 enzyme was measured as the percentage decrease in the activity of marker metabolite formation compared to non-inhibited controls. IC₅₀ values were determined on GraphPad Prism with remaining activity (%) and logarithm of inhibitor concentrations fitted with a non-linear fit [inhibitor] vs normalized response with variable slope.

### Determination of CYP2D6 Time-Dependent Inhibition by 15

Assessment of compound 15 was carried out at Pharmaron Inc. for potential time-dependent inhibition of CYP2D6 using human liver microsomes (BD Gentest) and primary human hepatocytes (BioIVT). Bufuralol (2 μM) was used as a substrate for liver microsomes and dextromethorphan (40 μM) for hepatocytes. The activities tested were CYP2D6-mediated bufuralol 1′-hydroxylation and CYP2D6-mediated dextromethorphan O-demethylation. Compound 15 was assayed in duplicate (n=2) and tested in a six-point, threefold dilution series (0.03 μM to 10 μM) in DMSO and incubated with either pooled human liver microsomes (0.5 mg/mL) or human hepatocytes (0.3×10⁶ cells/mL). Incubations in pooled human liver microsomes were carried out with NADPH (1 mM final concentration) with or without a 30 min pre-incubation at 37° C., 5% CO₂. Incubations in human hepatocytes were carried out without pre-incubation or with a 30 min pre-incubation at 37° C., 5% CO₂. The experiment was carried out at 37° C., 5% CO₂ for 5 min. Inhibition of CYP2D6 was measured as the percentage decrease in the activity of marker metabolite formation compared to non-inhibited controls. IC₅₀ values were determined on GraphPad Prism with remaining activity (%) and logarithm of inhibitor concentrations fitted with a non-linear fit [inhibitor] vs normalized response with variable slope.

### AMES Fluctuation Test (Eurofins Panlabs, St Charles, MO, USA)

Compounds were assessed for mutagenicity at Eurofins Panlabs. in 384-well plates using four Salmonella strains: TA98, to probe for frameshift mutation (with quercetin as control), TA100 and TA1535, to probe for base pair insertions/deletions (with streptozotocin as control), and TA1537, to probe for frameshift mutations (with aminoacridine as control) as previously described.²¹ Each compound was incubated at 37° C. for 96 h at four different concentrations (5, 10, 50, and 100 μM), with each concentration tested using 48 replicates, both with and without rat liver S9 metabolic activation. A concurrent bacterial cytotoxicity assay was ran at 0.6, 1.2, 2.5, 5, 10, 25, 50 and 100 μM to rule out false negatives. Bacterial cytotoxicity was expressed as percent of control growth (OD₆₅₀). Compounds with growth of less than 60% control were flagged and considered bacterial cytotoxic. Wells that displayed bacterial growth due to the reversion of the histidine mutation (as judged by the ratio of OD₄₃₀/OD₅₇₀ being greater than 1.0) were counted and recorded as positive counts. Significances of the positive counts between the treatment and control were determined using one-tailed Fisher's exact test.

### In Vitro Micronucleus Test

Compound 15 was assessed for genotoxicity by Eurofins Panlabs. using the in vitro micronucleus test in Chinese hamster ovary (CHO-K1) cells as previously described.²² Compound 15 was incubated in a 96-well plate format at varying concentrations (8, 16, 31, 62, 125, 250, 500 and 1000 μM) for 4 h at 37° C. with metabolic activation by rat liver S9 and 24 h at 37° C. without metabolic activation by rat liver S9. High content analysis and fluorescence imaging were used to detect micronuclei and compared against positive controls (cyclophosphamide (ran at 7.2 μM) and mitomycin C (ran at 0.3 μM)). Significances of the positive counts between the treatment and control were determined by one-tailed t-test with two sample equal variance.

### Kinase Selectivity Determination of 15

Profiling of a 468-member human kinase panel was performed at Eurofins DiscoverX using the KINOMEScan platform. A panel of 468 kinases was assayed at a single concentration of 1 μM for 15. Percent control was mapped onto the kinome tree using TREEspot™. The S scores were calculated as previously described²³ and are reflective of the number of kinases bound by 15 over the total number of wild-type kinases.

### In Vitro CTG Assay (Crown Bioscience)

### Cell Lines

The A431, HCC827, SK-BR-3, ZR-75-30, AU565 and Caco-2 cell lines were obtained from ATCC. The NCI-H1975 cell line was obtained from SIBS. The NCI-H3255 cell line was obtained from CoBioer. Engineered Ba/F3 cell lines were obtained from Crown Bioscience. MCKII-MDR1 cells were obtained from the Netherlands Cancer Institute. HEK293 cells were obtained from Invitrogen. A431 cells were cultured in DMEM (Life Technologies) with 10% FBS. HCC827, NCI-H1975 and AU565 cells were cultured in RPMI1640 (Invitrogen) with 10% FBS. NCI-H3255 cells were cultured in BEGM (Lonza) with 10% FBS. Ba/F3 EGFR-del E746_A750/C797S and Ba/F3 EGFR-L858R/C797S cells were cultured in RPMI (Invitrogen) with 10% FBS. SK-BR-3 cells were cultured in McCoy's 5a (Invitrogen) with 10% FBS. ZR-75-30 cells were cultured in RPMI1640 (Invitrogen) with 20% FBS. BT474 cells were cultured in DMEM (Gibco) with 10% FBS and 10 μg/mL Insulin. Caco-2 and MCKII-MDR1 culture information is given in the permeability studies section. HEK293 cells were cultured in DMEM (Gibco) with 10% FBS, 0.1 mM NEAA, 25 mM HEPES, 100 U/mL penicillin-streptomycin, μg/mL blasticidin and 400 μg/mL geneticin. All cells were cultured in a humidified incubator with 5% CO₂ at 37° C.

Viability assays using A431, HCC827, NCI-H1975, NCI-H3255, Ba/F3 EGFR-del E746_A750/C797S, Ba/F3 EGFR-L858R/C797S, AU565, SK-BR-3 and ZR-75-30 cells were performed at Crown BioScience. Cells were plated into 96-well plates at 1,500-7,000 cells per well and dosed in triplicate (n=3) in a nine-point, fourfold dilution series with compounds (0.15 nM to 10 μM) in DMSO and incubated for 72 h. After 72 h, cell viability was assayed by CellTiter-Glo Luminescent Viability Assay (Promega). Dose-response curves were generated and used to calculate the IC₅₀ values which were calculated on GraphPad Prism from the non-linear regression equation fitted with a sigmoidal dose response and are presented as the mean±SEM.

### Animal Studies

Animal experiments were performed at Pharmaron Inc. and animal use was approved by Pharmaron's Institutional Animal Care and Use Committee (IACUC) in Pharmaron (Pharmaron IACUC, Protocol #PK-M-07182022, #PK-R-06012022 and #ON-CELL-XEN-06012022) following the guidance of AAALAC. Six- to eight-week-old male SD rats (approximately 200-300 g) obtained from Si Bei Fu Laboratory Animal Technology Co., six- to eight-week-old male CD1 mice (approximately 20-30 g) obtained from Si Bei Fu Laboratory Animal Technology Co., and six- to eight-week-old female BALB/c nude mice (approximately 20-30 g) obtained from Si Bei Fu Laboratory Animal Technology Co., were used in the pharmacokinetic studies. Six- to eight-week-old female BALB/c nude mice (approximately 18-22 g) obtained from Beijing Anikeeper Biotech Co., Ltd were used in the intracranial PDX study. Animals were housed at 20-25° C. with humidity ranging from 40-70% relative humidity, were exposed to 12 h light and dark cycles, and were supplied with food and water ab libitum.

### In Vivo Studies

### Pharmacokinetic Studies of 15

Standard pharmacokinetic assessment of 15 was done by IV (intravenous) tail vein injection (formulation: DMSO:10% captisol in saline=1:99) and PO (oral) oral gavage (formulation: 1% methylcellulose) followed by blood sampling at 8 time points for IV (0.0833, 0.25, 0.5, 1,2, 4, 7, 24 h post dose) and 7 time points for PO (0.25, 0.5, 1, 2, 4, 8, 24 h post dose) with n=3 animals per dosing route (n=6 total). Approximately 0.03 mL of blood was collected from the dorsal metatarsal vein (in the case of CD1 mice), 0.03 mL of blood from the orbit vein (in the case of BALB/c nude mice) or 0.2 mL of blood from the jugular vein (in the case of SD rats) at each time point. Blood at each sampling point was transferred into a plastic micro centrifuge tube containing K₂-EDTA and collection tubes with blood samples and anticoagulant were inverted several times for proper mixing of the tube contents and placed on ice prior to centrifugation for plasma. Blood samples were centrifuged at 4° C., 4,000 g for 5 min to obtain plasma. Samples were stored in a freezer at −75° C. prior to analysis. Concentrations of test articles in the plasma samples were determined using LC-MS/MS and WinNonlin 8.3 (Phoenix™) was used for pharmacokinetic calculations. The values obtained were plotted on GraphPad Prism software and are presented as the mean±SD.

Oral CNS pharmacokinetic assessment of 15 was done by oral gavage (formulation: 1% methylcellulose) followed by blood and brain sampling at 7 time points (0.25, 0.5, 1, 2, 4, 8, 24 h post dose) with n=3 animals per time point (n=21 total). For plasma collection, approximately 0.03 mL of blood was collected from the dorsal metatarsal vein (in the case of CD1 mice) or 0.2 mL of blood from the jugular vein (in the case of SD rats) at each time point. Blood at each sampling point was transferred into a plastic micro centrifuge tube containing K₂-EDTA and collection tubes with blood samples and anticoagulant were inverted several times for proper mixing of the tube contents and placed on ice prior to centrifugation for plasma. Blood samples were then centrifuged at 4° C., 4,000 g for 5 min to obtain plasma and the samples were stored in a freezer at −75° C. prior to analysis. For brain sample collection, rodents were fully exsanguinated prior to collection and the thoracic cavity was opened exposing the heart which was catheterized from the left ventricular. A small incision was then made at the right atrial appendage and a gentle administration of saline via syringe was performed. Brain samples were collected at each time point and quickly frozen in an ice box. Samples were stored at −75° C. prior to analysis. All brain samples were prepared with water to achieve a brain weight (g) to water volume (mL) ratio of 1:4 prior to analysis. Concentrations of 15 in the plasma and brain samples were determined using LC-MS/MS and WinNonlin 8.3 (Phoenix™) was used for pharmacokinetic calculations. The values obtained were plotted on GraphPad Prism software and are presented as the mean±SD.

### Intracranial PDX Model of 15

HCC827 cells stably expressing luciferase (HCC827-luc) were injected intracranially. Briefly, 3×10⁵ HCC827-luc tumor cells suspended in 2 μL RPMI1640 medium were injected into the right forebrain of anesthetized mice (anesthetic: intramuscular injection of Zoletl™ 50 (Virbac S.A)). Mice were imaged bi-weekly using IVIS Lumina Ill (Perkin Elmer). Images were acquired 10 min post intraperitoneal (IP) injection with 15 mg/mL (at 5 μL/g body weight) of D-luciferin in anesthetized mice (anesthetic: 1-2% isoflurane inhalation). On day 20 post-cellular inoculation, mice were randomized into 2 treatment groups (n=10 mice per group): 1) 1% methylcellulose as vehicle control and 2) 15 (10 mg/kg) (formulation: 1% methylcellulose), PO, b.i.d. (twice daily) dosing (12 h intervals)×7 days for 21 days. Mice were to be euthanized under the following circumstances: 1) if the individual animal showed obvious signs of severe distress and/or pain, 2) if body weight (BW) loss exceeded 20% or 3) if animals were not able to get adequate food or water. Body weights of all mice were measured bi-weekly throughout the study and BW change, expressed in % was calculated using the following formula.

BW change (%)=(BW_{Day X}/BW_{Day 0})×100

Where, BW_{Day X} is the BW on a given day, and BW_{Day 0} is BW on Day 0 (initiation of treatment).

### Statistical Analyses

Statistical analysis was performed using GraphPad Prism 9.0. Data are presented as mean t SD or SEM as indicated when n=3, or as the mean when n=2. For in vitro ADME and kinase activity studies, data is presented as mean of n=2 independent replicates. For in vivo PK experiments, data is presented as the mean±SD, n 3 animals per study arm. For in vitro short-term growth delay experiments, IC₅₀ values were determined from the nonlinear regression equation fitted with a sigmoidal dose response curve and are presented as the mean±SEM, n=3 independent replicates. Intracranial PDX study data (bioluminescence and body weight means on day-21 post-treatment initiation, n=10 animals per study arm) were compared using a two-tailed unpaired t-test. A p value of <0.05 was considered statistically significant.

### Results and Discussion

As shown in FIG. 4, hydroxalog (6) was prepared using our direct N—O bond forming reaction.¹¹ To this end, the 2-methyltetrahydropyranyl (MTHP) monoperoxyacetal¹¹⁻¹³ (8) derived from commercially available 2-((tert-butyldimethylsilyl)oxy)ethanol (7) in 51% yield, was exposed to a morpholine derived magnesium amide affording the hydroxylamine (10) in 65% yield on over an 8 gram scale. Notably, we found that the use of turbo-Grignard (ⁱPrMgCl·LiCl) developed by Knochel²⁴ proved optimal for N—O bond formation on larger scale. While generation of the morpholine derived magnesium amide from EtMgBr afforded products in reproducible yields on small scale, on larger scale (>5 gram) this magnesium amide displayed solubility issues thus prompting us to investigate alternatives (see Examples section for more details). Nevertheless, desilylation of 10 in 79% yield afforded alcohol (11), which was converted to the chloride by reaction with thionyl chloride and subsequently displaced with commercially available 4-(3-chloro-4-fluorophenylamino)-7-methoxyquinazolin-6-ol under basic conditions affording the intended hydroxalog (6) in 35% yield over 2 steps. Overall, hydroxalog (6) was prepared in only 4 longest-linear steps (LLS) from readily available starting materials, highlighted by direct N—O bond formation with all synthetic transformations carried out on gram-scale.

With 6 in hand, we began by evaluating inhibitor-binding constants and biochemical inhibition of the relevant EGFR kinase forms of 1 and 6, a direct hydroxylamine analog; both 1 and 6 exhibited single-digit nanomolar activity against activating mutant bearing EGFR, indicating that an N-(noralkoxy)morpholine unit is an effective bioisostere of the N-alkylmorpholine unit. We then assessed in vitro ADME properties, beginning with a colon-carcinoma (Caco-2) cell permeability assay, which expresses both P-gp and BCRP; 6 displayed a 14-fold enhancement in permeability and greatly decreased efflux compared to 1 (TABLE 1). Substantiating this effect, 6 also showed a roughly 4-fold increase in permeability and decreased efflux compared to 1 in a Madin-Darby canine kidney (MDCK) MDCKII-MDR1 cell permeability assay, that is commonly used to mimic the BBB through overexpression of the active efflux transporter P-gp, or MDR1. These results indicated that replacement of the N-alkylmorpholine unit by an N-(noralkoxy)morpholine unit might overcome the known²⁵ poor BBB permeability and active efflux observed with 1. Of note, currently osimertinib (5), a third-generation EGFR TKI is the only approved EGFR TKI that shows promise in treating BM in EGFR+ NSCLC despite it being a substrate for both P-gp and BCRP.⁸ We then performed an AMES fluctuation assay both with and without metabolic activation by rat liver S9 (+/−S9) across 4 Salmonella strains (TA98, TA100, TA1537 and TA1535), and found that neither 1 or 6 were mutagenic.²⁶ Taken in combination with a similar stability profile across multiple species in liver microsomes and plasma as compared to 1, these findings dispelled any early concerns regarding perceived mutagenicity and instability towards oxidative metabolism of the trisubstituted hydroxylamine unit in 6.

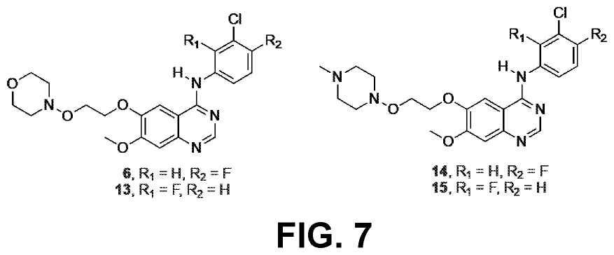

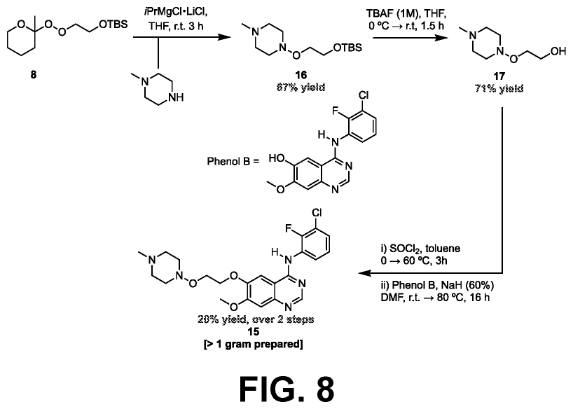

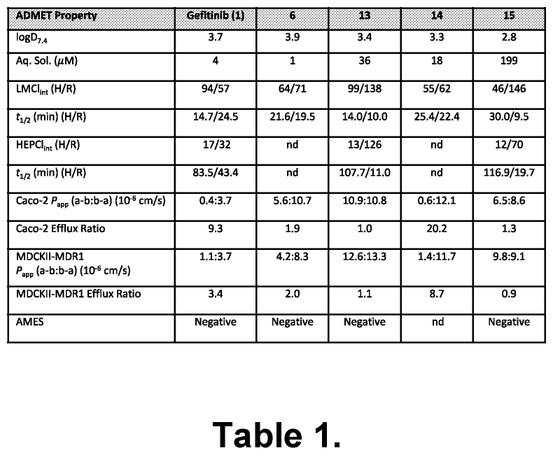

We sought to further minimize efflux and improve drug-like properties, particularly the aqueous solubility of 6, by synthesizing additional trisubstituted hydroxylamine-bearing inhibitors (vide infra) (FIGS. 7-8). In view of the importance of hydrogen bond donors (HBD) on efflux transporter substrate recognition, and noting the improved CNS penetrability of EGFR inhibitors AZD3759 and JCN037, 13 was designed with a para- to ortho-fluorine switch to minimize the HBD capability of the adjacent aniline (FIG. 7).²⁷⁻³⁰ Additionally, 14 and 15 were prepared by exchange of the morpholine unit with an N-methylpiperazine group, all while maintaining the key trisubstituted hydroxylamine moiety, with the intent of improving aqueous solubility and potential in vivo exposure. All newly prepared analogs maintained single digit to sub-nanomolar binding and biochemical inhibition of activating mutant bearing EGFR. In the Caco-2 cellular assay, 13 and 15, which bear the ortho-fluorine, exhibited remarkably enhanced permeability and reduced efflux ratios compared to 1 and even improved to that seen with 6 (TABLE 1). Notably 14, that contains a more basic nitrogen heterocycle than that of 1, 6 and 13 and a para-fluoro substitution pattern on the aniline ring, showed high efflux and low permeability, highlighting the synergistic improvement of modified HBD and reduced pKa on efflux transporter substrate recognition. In the MDCKII-MDR1 cellular assays, 13 and 15 showed excellent permeability and low efflux as compared to 1, 14 and even 6. Importantly, 15 also displayed good stability in human and rat microsomes and hepatocytes while exhibiting markedly improved aqueous solubility compared to both 1 and 6.

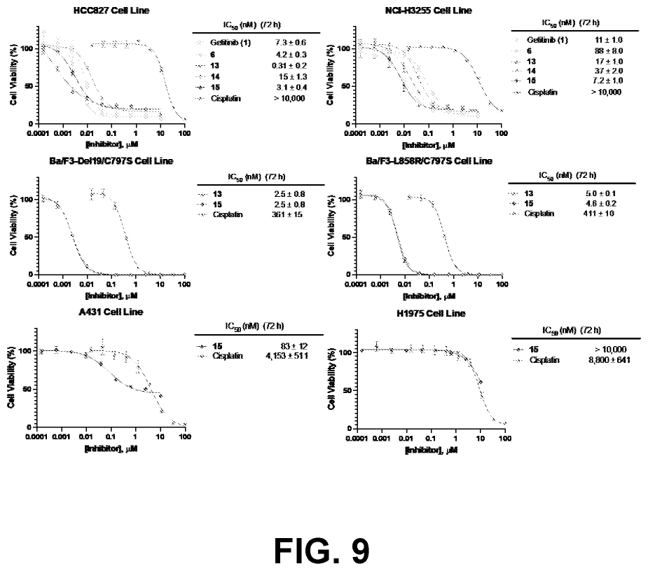

We profiled the anticancer activity of the hydroxylamine-bearing inhibitors against four patient-derived cell lines harboring differing EGFR status with cisplatin as the positive control throughout (FIG. 9). Notably, 15 displayed excellent activity against the NCI-H3255 NSCLC cell line bearing the common EGFR^{L858R} mutation, with an IC₅₀ of 7.2 nM representing a roughly 12-fold improvement over 6. Potent activity of 15 was also seen in HCC827 NSCLC cells harboring EGFR^{del E746_A750} with an IC₅₀ of 3.1 nM. In osimertinib-resistant engineered Ba/F3 cell lines bearing EGFR^{L858R/C797S} and EGFR^{del E746_A750/C797S} which block covalent inhibitor binding by a C797S point mutation, 15 also exhibited strong activity, with IC₅₀ values of 4.6 and 2.5 nM, respectively.³¹ In the skin-derived A431 cell line bearing overexpressed EGFR^{wt} 15 exhibited a comparatively high IC₅₀ of 83 nM, affording 12- to 42-fold selectivity for activating mutant EGFR over wild-type EGFR. Overall, 15 is a potent inhibitor in osimertinib-resistant engineered cell lines and patient-derived NSCLC cell lines NCI-H3255 and HCC827, which harbor mutations in EGFR that encompass approximately 85% of all newly diagnosed mutant EGFR+ NSCLC cases.

Moving forward with 15, we determined the unbound fractions in plasma and brain tissue (TABLE 2).¹⁰ Both 15 and 1 exhibited similar plasma protein binding, however, 15 showed an excellent unbound rat brain tissue fraction (f_{u,brain} %=4.5) compared to 1 (f_{u,brain} %=0.6). With respect to potential toxicity, only moderate human Ether-à-go-go-Related Gene (hERG) potassium ion channel inhibition by 15 was observed with an IC₅₀ of 6.48 μM, and a maximal inhibition of approximately 60% at 10 μM (TABLE 2). We also assayed for CYP inhibition across all major isoforms (3A4, 1A2, 2C9, 2D6, 2C19) and saw none (IC₅₀=>30 μM) except for moderate inhibition of CYP2D6 (IC₅₀=1.1 μM) (TABLE 2). In a follow-up CYP2D6-time-dependent inhibition (TDI) IC₅₀-shift experiment in human liver microsomes and primary human hepatocytes, this latter activity was shown not to be time dependent, thus dispelling concerns of potential drug-drug interactions (DDI).³² In the AMES fluctuation assay across 4 Salmonella strains (TA98, TA100, TA1537 and TA1535), and an in vitro micronucleus test in Chinese hamster ovary (CHO-K1) cells both with and without metabolic activation by rat liver S9 (+/−S9), 15 exhibited neither mutagenic nor genotoxic potential, contrary to the popular belief that hydroxylamines are inherently mutagenic and genotoxic (TABLE 2)^{33-34, 26, 35} Overall, these experiments indicate that 15 is a potent inhibitor of activating mutant bearing EGFR and is devoid of the efflux liabilities that characterize currently approved targeted EGFR therapies.

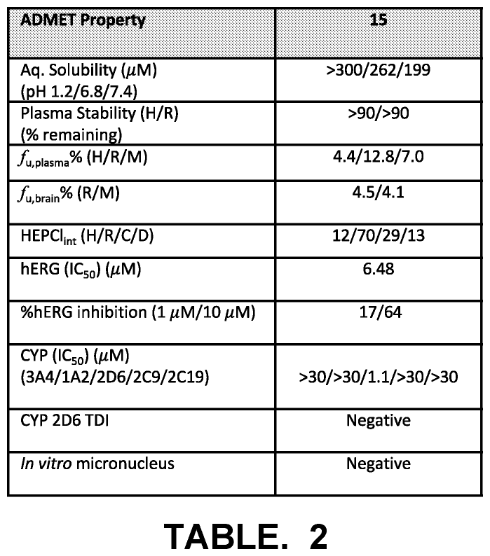

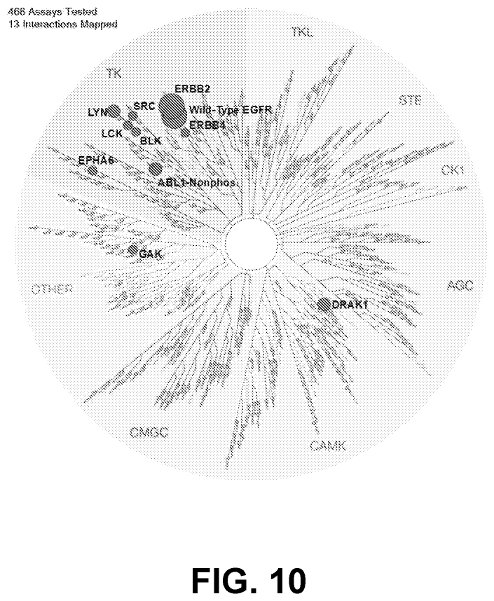

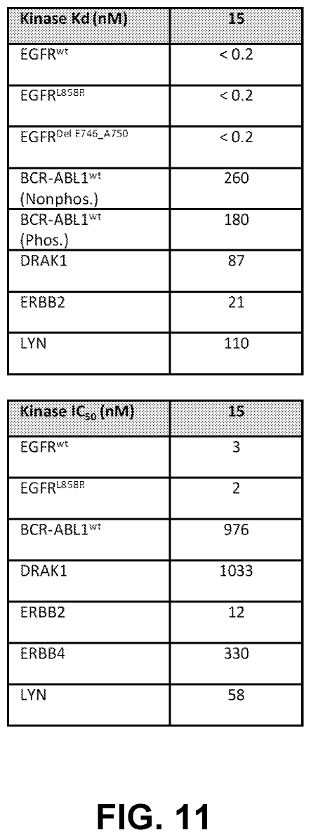

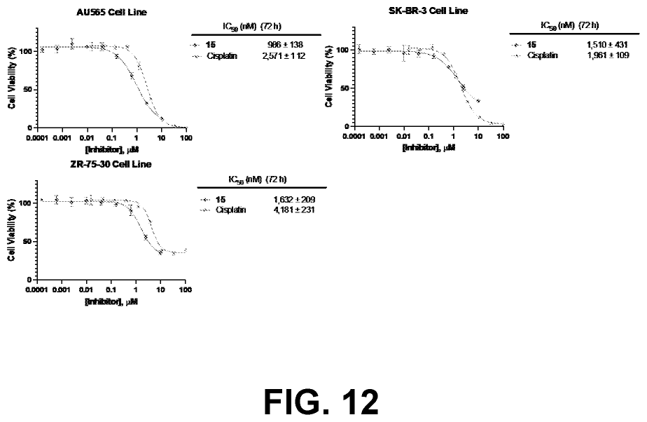

Using KINOMEScan technology, we determined the selectivity of 15 against a panel of >400 human kinases at a concentration of 1 μM; exquisite kinase selectivity was found with an S(10) score of 0.015 (6/403 nonmutant kinases showing 10% activity at 1 μM) (FIG. 10).²³ Apart from EGFR^{wt} (0.30%), 15 only showed high affinity to ERBB2 (HER2) (0.45%). Moderate affinity for ABL1 (7.7%), DRAK1 (8.3%), LYN (7.4%), and PIKFYVE (1.6%) was also observed, but confirmed in a follow-up binding experiment to be of minor significance considering the potent binding to EGFR (K_{d}=<0.2 nM) (FIG. 11). As a result of the moderate affinity for HER2, that was confirmed in a follow up binding experiment (K_{d}=21 nM) and considering the lack of efficient treatments for metastatic HER2+ breast cancer,³⁶ we further profiled 15 against HER2 using additional biochemical and cellular assays (FIG. 12). We found that 15 exhibited low antiproliferative activity in HER2+ breast cancer cell lines in comparison to activating mutant bearing EGFR+ cells, with IC₅₀'s ranging from 0.99 to 1.6 μM.

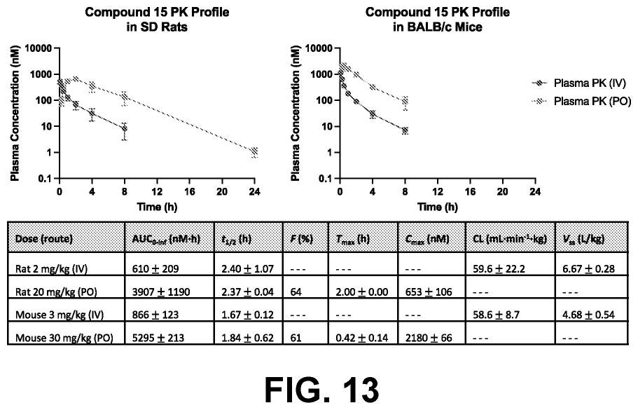

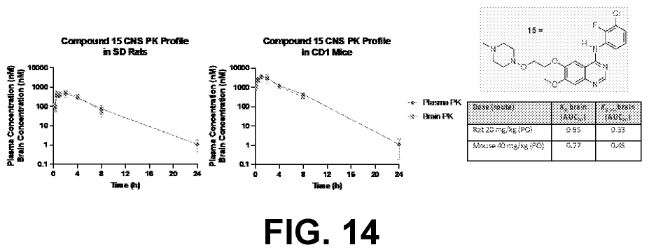

To qualify 15 as a candidate for further studies, we evaluated its pharmacokinetic properties after administration into Sprague Dawley (SD) rats (FIG. 13). No adverse events or signs of toxicity were observed for the compound at the tested doses, contrary to the common belief that hydroxylamines are inherently toxic.³³⁻³⁴ Moreover, 15 showed high oral bioavailability (F=64%), exposure (AUC_{inf}=3907 h-ng/mL) and an acceptable half-life (t_{1/2}=2.37 h) with a high volume of distribution (Vₛₛ=6.67 L/kg) at 2 mg/kg intravenous (IV) and 20 mg/kg oral (PO) dosing in SD rats (FIG. 13). After administration of a single oral dose of 20 mg/kg in SD rats, 15 exhibited a K_{p,uu} (unbound brain-to-unbound plasma partitioning coefficient) (AUC_{inf}) of 0.33 and a Kₚ (brain-to-plasma partitioning coefficient) (AUC_{inf}) of 0.95, indicating excellent brain penetration (FIG. 14)^{10,37} Substantiating this effect, after administration of a single oral dose of 40 mg/kg in CD1 mice, a K_{p,uu} (AUC_{inf}) of 0.45 and a Kₚ (AUC_{inf}) of 0.77 was obtained (FIG. 14).

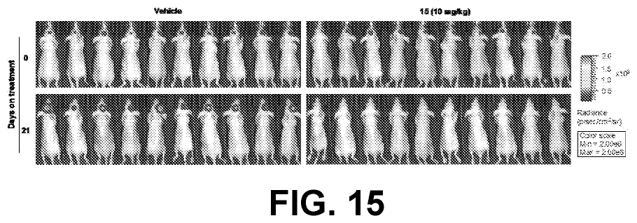

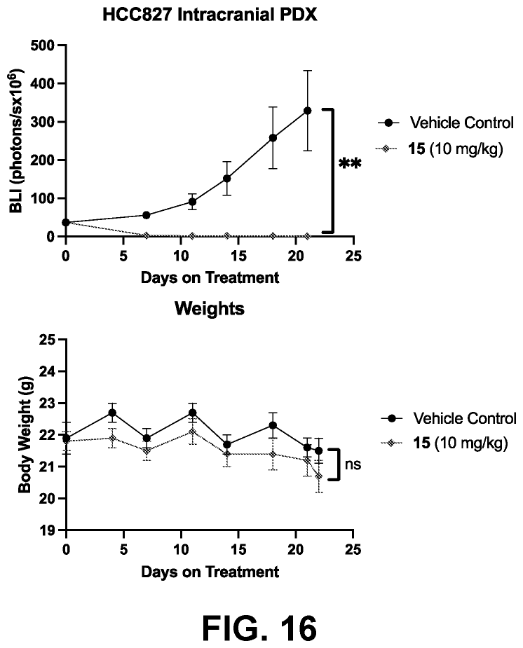

Finally, to assess the viability of 15 as a potential treatment for BM in EGFR+ NSCLC, we performed an intracranial patient-derived xenograft (PDX) model with luciferase-tagged HCC827 cells implanted into the brains of BALB/c nude mice (FIG. 15). At 10 mg/kg PO b.i.d (twice daily) dosing, profound tumor regression (P=0.0059) relative to vehicle control (1% methylcellulose) was observed, confirming that 15 has intracranial antitumor activity (FIG. 16). Additionally, over the 21-day treatment window, mean body weight loss never exceeded 10% and no adverse clinical events were observed at the studied dose, thereby demonstrating the potential of 15 as a candidate for CNS metastatic EGFR+ NSCLC (FIG. 16).

### CONCLUSIONS

A highly selective, orally bioavailable, brain-penetrant EGFR inhibitor, 15, bearing a novel amine bioisostere, the trisubstituted hydroxylamine, is described. In contrast to the common expectation for hydroxylamines in medicinal chemistry, 15 lacks mutagenic or genotoxic potential and exhibits good stability in vitro and in vivo. An intracranial PDX murine model revealed profound tumor regression on oral dosing with 15 suggesting this novel compound as a potential lead in the treatment of localized and CNS metastatic NSCLC driven by activating mutant bearing EGFR and for osimertinib resistant EGFR+ NSCLC. All told, our results show that the trisubstituted hydroxylamine moiety, a “structural alert” in medicinal chemistry, can be incorporated into drug scaffolds to improve drug properties, while maintaining potent biological activity and avoiding molecular weight creep. These findings support broader application of trisubstituted hydroxylamines as bioisosteres in drug discovery programmes for lead optimization and in patent life-cycle management.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Thai, A. A.; Solomon, B. J.; Sequist, L. V.; Gainor, J. F.; Heist, R. S. Lung cancer. Lancet 2021, 398 (10299), 535-554.
2. Peaz, J. G.; Jänne, P. A.; Lee, J. C.; Tracy, S.; Greulich, H.; Gabriel, S.; Herman, P.; Kaye, F. J.; Lindeman, N.; Boggon, T. J.; Naoki, K.; Sasaki, H.; Fujii, Y.; Eck, M. J.; Sellers, W. R.; Johnson, B. E.; Meyerson, M. EGFR mutations in lung cancer: correlation with clinical response to gefitinib therapy. Science 2004, 304 (5676), 1497-1500.
3. Lynch, T. J.; Bell, D. W.; Sordella, R.; Burubhagavatula, S.; Okimoto, R. A.; Brannigan, B. W.; Harris, P. L.; Haserlat, S. M.; Supko, J. G.; Haluska, F. G.; Louis, D. N.; Christiani, D. C.; Settleman, J.; Haber, D. A. Activating mutations in the epidermal growth factor receptor underlying responsiveness of non-small-cell lung cancer to gefitinib. N. Engl. J. Med. 2004, 350 (21), 2129-2139.
4. Pao, W.; Miller, V.; Zakowski, M.; Doherty, J.; Politi, K.; Sarkaria, I.; Singh, B.; Heelan, R.; Rusch, V.; Fulton, L.; Mardis, E.; Kupfer, D.; Wilson, R.; Kris, M.; Varmus, H. EGF receptor gene mutations are common in lung cancers from “never smokers” and are associated with sensitivity of tumors to gefitinib and erlotinib. Proc. Natl. Acad. Sci. U.S.A. 2004, 101 (36), 13306-13311.
5. Rosell, R.; Moran, T.; Queralt, C.; Porta, R.; Cardenal, F.; Camps, C.; Majem, M.; Lopez-Vivanco, G.; Isla, D.; Provencio, M.; Insa, A.; Massuti, B.; Gonzalez-Larriba, J. L.; Paz-Ares, L.; Bover, I.; Garcia-Campelo, R.; Moreno, M. A.; Catot, S.; Rolfo, C.; Reguart, N.; Palmero, R.; Sanchez, J. M.; Bastus, R.; Mayo, C.; Bertran-Alamillo, J.; Molina, M. A.; Sanchez, J. J.; Taron, M. Screening for epidermal growth factor receptor mutations in lung cancer. N. Engl. J. Med. 2009, 361 (10), 958-967.
6. Nayak, L.; Lee, E. Q.; Wen, P. Y. Epidemiology of brain metastases. Curr. Oncol. Rep. 2012, 14 (1), 48-54.
7. Rangachari, D.; Yamaguchi, N.; VanderLaan, P. A.; Folch, E.; Mahadevan, A.; Floyd, S. R.; Uhlmann, E. J.; Wong, E. T.; Dahlberg, S. E.; Huberman, M. S.; Costa, D. B. Brain metastases in patients with EGFR-mutated or ALK-rearranged non-small-cell lung cancers. Lung Cancer. 2015, 88 (1), 108-111.
8. Coclough, N.; Chen, K.; Johnström, P.; Strittmatter, N.; Yan, Y.; Wrigley, G. L.; Schou, M.; Goodwin, R.; Varnäs, K.; Adua, S. J.; Zhao, M.; Nguyen, D. X.; Maglennon, G.; Barton, P.; Atkinson, J.; Zhang, L.; Jandefeldt, A.; Wilson, J.; Smith, A.; Takano, A.; Arakawa, R.; Kondrashov, M.; Malmquist, J.; Revunov, E.; Vazquez-Romero, A.; Moein, M. M.; Windhorst, A. D.; Karp, N. A.; Finlay, M. R. V.; Ward, R. A.; Yates, J. W. T.; Smith, P. D.; Farde, L.; Cheng, Z.; Cross, D. A. E. Preclinical comparison of the blood-brain barrier permeability of osimertinib with other EGFR TKIs. Clin. Cancer Res. 2021, 27 (1), 189-201.
9. Heon, S.; Yeap, B. Y.; Britt, G. J.; Costa, D. B.; Rabin, M. S.; Jackman, D. M.; Johnson, B. E. Development of central nervous system metastases in patients with advanced non-small cell lung cancer and somatic EGFR mutations treated with gefitinib or erlotinib. Clin. Cancer Res. 2010, 16 (23), 5873-5882.
10. Heffron, T. P. Small molecule kinase inhibitors for the treatment of brain cancer. J. Med. Chem. 2016, 59 (22), 10030-10066.
11. Hill, J.; Hettikankanamalage, A. A.; Crich, D. Diversity-oriented synthesis of N,N,O-trisubstituted hydroxylamines from alcohols and amines by direct N—O bond formation. J. Am. Chem. Soc. 2020, 142 (35), 14820-14825.
12. Hoang, K. M.; Lees, N. R.; Herzon, S. B. Programmable synthesis of 2-deoxyglycosides. J. Am. Chem. Soc. 2019, 141 (20), 8098-8103.
13. Hoang, K. M.; Lees, N. R.; Herzon, S. B. General method for the synthesis of α- or β-deoxyaminoglycosides bearing basic nitrogen. J. Am. Chem. Soc. 2021, 143 (7), 2777-2783.
14. Hill, J.; Crich, D. The N,N,O-trisubstituted hydroxylamine isostere and its influence on lipophilicity and related parameters. ACS Med. Chem. Lett. 2022, 13 (5), 799-806.
15. Hill, J.; Beckler, T. D.; Crich, D. Recent advances in the synthesis of di- and trisubstituted hydroxylamines. Molecules. 2023, 28 (6), 2816.
16. Meisenheimer, J. Über eine eigenartige Umlagerung des Methyl-allyl-anilin-N-oxyds. Ber. Dtsch. Chem. Ges. 1919, 52, 1667-1677.
17. Albini, A. Synthetic Utility of amine N-oxides. Synthesis 1993, 3, 263-277.
18. Vallakati, R.; May, J. A. Biomimetic synthesis of the antimalarial flindersial alkaloids. J. Am. Chem. Soc. 2012, 134 (16), 6936-6939.
19. Kalvass, J. C.; Tess, D. A.; Giragossian, C.; Linhares, M. C.; Maurer, T. S. Influence of microsomal concentration on apparent intrinsic clearance: implications for scaling in vitro data. Drug Metab. Dispos. 2001, 29 (10), 1332-1336.
20. Danker, T.; Möller, C. Early identification of hERG liability in drug discovery programs by automated patch clamp. Front Pharmacol. 2014, 5, 1-11.
21. Maron, D. M.; Ames, B. N. Revised methods for the Salmonella mutagenicity test. Mutat. Res. 1983, 113, 173-215.
22. Diaz, D.; Scott, A.; Carmichael, P.; Shi, W.; Costales, C. Evaluation of an automated in vitro micronucleus assay in CHO-K1 cells. Mutat. Res. 2007, 630, 1-13.
23. Davis, M. I.; Hunt, J. P.; Herrgard, S.; Ciceri, P.; Wodicka, L. M.; Pallares, G.; Hocker, M.; Treiber, D. K.; Zarrinkar, P. P. Comprehensive analysis of kinase inhibitor selectivity. Nat. Biotechnol. 2011, 29 (11), 1046-1051.
24. Krasovskiy, A.; Knochel, P. A LiCl-mediated Br/Mg exchange reaction for the preparation of functionalized aryl- and heteroarylmagnesium compounds from organic bromides. Angew. Chem. Int. Ed. 2004, 43 (25), 3333-3336.
25. Agarwal, S.; Sane, R.; Gallardo, J. L.; Ohlfest, J. R.; Elmquist, W. F. Distribution of gefitinib to the brain is limited by P-glycoprotein (ABCB₁) and breast cancer resistance protein (ABCG₂)-mediated active efflux. J. Pharmacol. Exp. Ther. 2010, 334 (1), 147-155.
26. Ames, B. N.; McCann, J.; Yamasaki, E. Methods for detecting carcinogens and mutagens with the Salmonella/mammalian-microsome mutagenicity test. Mutat. Res. 1975, 31 (6), 347-364.
27. Zeng, Q.; Wang, J.; Cheng, Z.; Chen, K.; Johnström, P.; Varnäs, K.; Li, D. Y.; Yang, Z. F.; Zhang, X. Discovery and evaluation of clinical candidate AZD3759, a potent, oral active, central nervous system-penetrant, epidermal growth factor receptor tyrosine kinase inhibitor. J. Med. Chem. 2015, 58 (20), 8200-8215.
28. Yang, Z.; Guo, Q.; Wang, Y.; Chen, K.; Zhang, L.; Cheng, Q.; Xu, Y.; Yin, X.; Bai, Y.; Rabbie, S.; Kim, D.-W.; Ahn, M.-J.; Yang, J. C.-H.; Zhang, X. AZD3759, a BBB-penetrating EGFR inhibitor for the treatment of EGFR mutant NSCLC with CNS metastases. Sci. Transl. Med. 2016, 8 (368), 368ra172.
29. Tsang, J. E.; Urner, L. M.; Kim, G.; Chow, K.; Baufeld, L.; Faull, K.; Cloughesy, T. F.; Clark, P. M.; Jung, M. E.; Nathanson, D. A. Development of a potent brain-penetrant EGFR tyrosine kinase inhibitor against malignant brain tumors. ACS Med. Chem. Lett. 2020, 11 (10), 1799-1809.
30. Weiss, M. W.; Williamson, T.; Babu-Khan, S.; Bartberger, M. D.; Brown, J.; Chen, K.; Cheng, Y.; Citron, M.; Croghan, M. D.; Dineen, T. A.; Esmay, J.; Graceffa, R. F.; Harried, S. S.; Hickman, D.; Hitchcock, S. A.; Horne, D. B.; Huang, H.; Imbeah-Ampiah, R.; Judd, T.; Kaller, M. R.; Kreiman, C. R.; La, D. S.; Li, V.; Lopez, P.; Louie, S.; Monenschein, H.; Nguyen, T. T.; Pennington, L. D.; Rattan, C.; Miguel, T. S.; Sickmier, E. A.; Wahl, R. C.; Wen, P. H.; Wood, S.; Xue, Q.; Yang, B. H.; Patel, V. F.; Zhong, W. Design and preparation of a potent series of hydroxyethylamine containing β-secretase inhibitors that demonstrate robust reduction of central β-amyloid. J. Med. Chem. 2012, 55 (21), 9009-9024.
31. Leonetti, A.; Sharma, S.; Minari, R.; Perego, P.; Giovannetti, E.; Tiseo, M. Resistance mechanisms to osimertinib in EGFR-mutated non-small cell lung cancer. Br. J. Cancer 2019, 121 (9), 725-737.
32. Grimm, S. W. Einolf, H. J.; Hall, S. D.; He, K.; Lim, H.-L.; Ling, K.-H. J.; Lu, C.; Nomeir, A. A.; Seibert, E.; Skordos, K. W.; Tonn, G. R.; Horn, R. V.; Wang, R. W.; Wong, Y. N.; Yang, T. J.; Obach, R. S. The conduct of in vitro studies to address time-dependent inhibition of drug-metabolizing enzymes: a perspective of the pharmaceutical research and manufacturers of America. Drug Metab. Dispos. 2009, 37 (7), 1355-1370.
33. Yum B.; Reynisson, J. Bond stability of the “undesirable” heteroatom-heteroatom molecular moieties for high-throughput screening libraries. Eur. J. Med. Chem. 2011, 46 (11), 5833-5837.
34. Bruns, B. F.; Watson, I. A. Rules for identifying potentially reactive or promiscuous compounds. J. Med. Chem. 2012, 55 (22), 9764-9772.
35. Corvi, R.; Albertini, S.; Hartung, T.; Hoffmann, S.; Maurici, D.; Pfuhler, S.; Benthem, J. V.; Vanparys, P. ECVAM retrospective validation of in vitro micronucleus test (MNT). Mutagenesis 2008, 23 (4), 271-283.
36. Winer, E. P.; Lin, N. U. Brain metastases: the HER2 paradigm. Clin. Cancer Res. 2007, 13 (6), 1648-1655.
37. Di, L., Rong, H.; Feng, B. Demystifying brain penetration in central nervous system drug discovery. J. Med. Chem. 2013, 56 (1), 2-12.

1. A compound having structure I or the pharmaceutically acceptable salt thereof 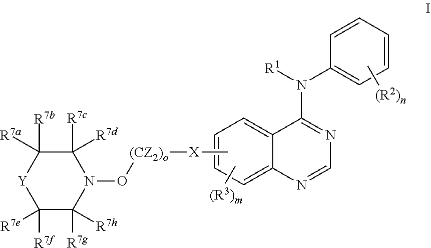
wherein
R¹ is hydrogen, a substituted or unsubstituted linear or branched alkyl group, a substituted or unsubstituted cycloalkyl group or heterocycloalkyl group, or a substituted or unsubstituted aryl group;
n is an integer from 1 to 5, where each R² is independently hydrogen, a substituted or unsubstituted linear or branched alkyl group, a substituted or unsubstituted cycloalkyl group or heterocycloalkyl group, a substituted or unsubstituted aryl group, a halide, or an alkoxy group;
m is an integer from 1 to 3, where each R³ is independently hydrogen, a substituted or unsubstituted linear or branched alkyl group, a substituted or unsubstituted cycloalkyl group or heterocycloalkyl group, a substituted or unsubstituted aryl group, a halide, or an alkoxy group;
o is an integer from 1 to 10;
X is O or NR⁴, wherein R⁴ is hydrogen, a substituted or unsubstituted linear or branched alkyl group, a substituted or unsubstituted cycloalkyl group or heterocycloalkyl group, or a substituted or unsubstituted aryl group;
Y is O, NR⁵, or CR^{6a}R^{6b}, wherein R⁵ is hydrogen, a substituted or unsubstituted linear or branched alkyl group, a substituted or unsubstituted cycloalkyl group or heterocycloalkyl group, or a substituted or unsubstituted aryl group, and
R^{6a} and R^{6b} are independently hydrogen, deuterium, a substituted or unsubstituted linear or branched alkyl group, a substituted or unsubstituted cycloalkyl group or heterocycloalkyl group, a substituted or unsubstituted aryl group, or a substituted or unsubstituted amino group;
R^{7a}, R^{7b}, R^{7c}, R^{7d}, R^{7e}, R^{7f}, R^{7g}, and R^{7h} are independently hydrogen, deuterium, or a substituted or unsubstituted alkyl group; and
each Z is independently hydrogen or deuterium.

2. The compound of claim 1, wherein X is O.

3. (canceled)

4. The compound of claim 1, wherein Y is O, NR⁵, where R⁵ is a C1 to C5 alkyl group, or CR^{6a}R^{6b}, where R^{6a} is hydrogen and R^{6b} is a substituted or unsubstituted amino group.

5. (canceled)

6. The compound of claim 4, wherein R¹ is hydrogen, R³ is an alkoxy group and m is 1, o is an integer from 1 to 5, and R² is a halide and n is 2.

7-23. (canceled)

24. The compound of claim 1, wherein the compound has the structure II, III, or the pharmaceutically acceptable salt thereof 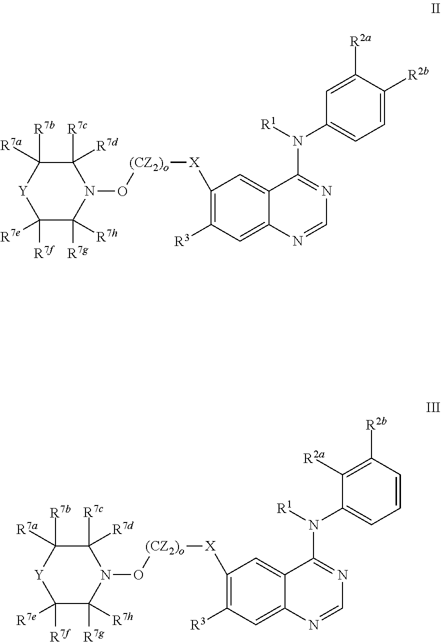
wherein
R¹ is hydrogen, a substituted or unsubstituted linear or branched alkyl group, a substituted or unsubstituted cycloalkyl group or heterocycloalkyl group, or a substituted or unsubstituted aryl group;
R^{2a} and R^{2b} are a halide;
R³ is an alkoxy group;
o is an integer from 1 to 5;
X is O or NR⁴, wherein R⁴ is hydrogen, a substituted or unsubstituted linear or branched alkyl group, a substituted or unsubstituted cycloalkyl group or heterocycloalkyl group, or a substituted or unsubstituted aryl group;
Y is O, NR⁵, or CR^{6a}R^{6b}, wherein R⁵ is hydrogen, deuterium, a substituted or unsubstituted linear or branched alkyl group, a substituted or unsubstituted cycloalkyl group or heterocycloalkyl group, or a substituted or unsubstituted aryl group, and
R^{6a} and R^{6b} are independently hydrogen, a substituted or unsubstituted linear or branched alkyl group, a substituted or unsubstituted cycloalkyl group or heterocycloalkyl group, a substituted or unsubstituted aryl group, or a substituted or unsubstituted amino group;
R^{7a}, R^{7b}, R^{7c}, R^{7d}, R^{7e}, R^{7f}, R^{7g}, and R^{7h} are independently hydrogen, deuterium, or a substituted or unsubstituted alkyl group; and
each Z is independently hydrogen or deuterium.

25. The compound of claim 24, wherein X is O.

26. (canceled)

27. The compound of claim 24, wherein Y is O, NR⁵, where R⁵ is a C1 to C5 alkyl group, or CR^{6a}R^{6b}, where R^{6a} is hydrogen and R^{6b} is a substituted or unsubstituted amino group.

28. (canceled)

29. The compound of claim 24, wherein R¹ is hydrogen.

30. The compound of claim 24, wherein R³ is a C1 to C10 substituted or unsubstituted linear or branched alkoxy group.

31. The compound of claim 24, wherein R³ is a methoxy group.

32. The compound of claim 24, wherein o is an integer from 1 to 5.

33. The compound of claim 24, wherein R² is a halide.

34. The compound of claim 24, wherein R^{2a} is chloride and R^{2b} is fluoride.

35. The compound of claim 24, wherein R^{2a} is fluoride and R^{2b} is chloride.

36. The compound of claim 1, wherein the compound is

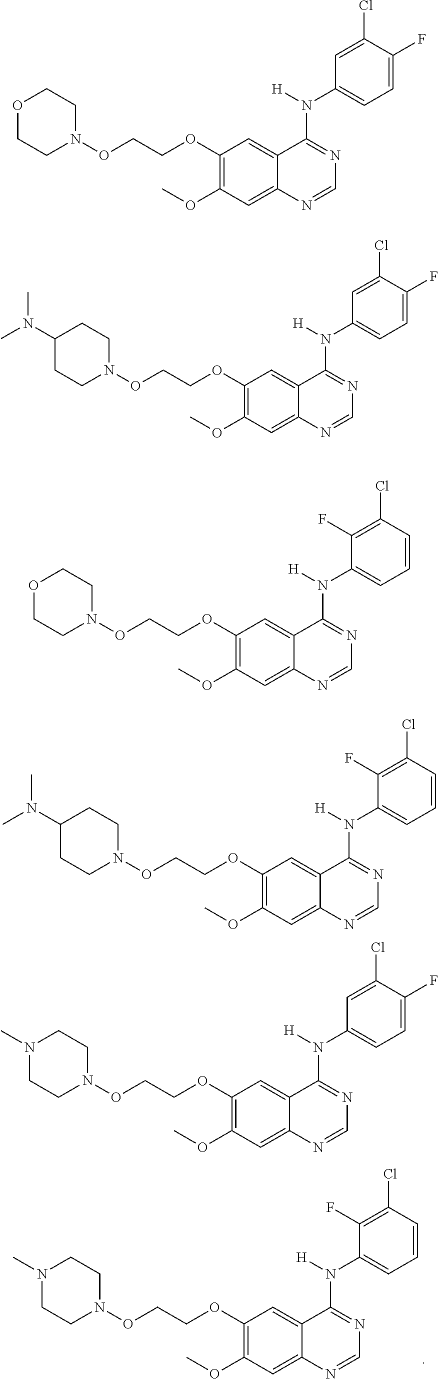

37. A pharmaceutical composition comprising the compound of claim 1 and a pharmaceutically-acceptable carrier.

38. A method for treating a subject having non-small cell lung cancer, neuroblastoma, glioblastoma multiforme, metastatic brain cancer, brain cancer, prostate cancer, or breast cancer, the method comprising administering to the subject an effective amount of the compound of claim 1.

39. A method for treating a subject having osimertinib-resistant cancer, the method comprising administering to the subject an effective amount of the compound of claim 1.

40. A method for treating a subject having brain metastases, the method comprising administering to the subject an effective amount of the compound of claim 1.

41. A method for inhibiting epidermal growth factor receptor (EGFR) in a subject, the method comprising administering to the subject an effective amount of the compound of claim 1.

42-100. (canceled)
